(19) United States
(12) Patent Application Publication
(10) Pub. No.: US 2024/0339217 A1
(43) Pub. Date: Oct. 10, 2024

(54) Medical Condition Visual Search

(71) Applicant: Google LLC, Mountain View, CA (US)

(72) Inventors: Peggy Yen Phuong Bui, San Francisco, CA (US); Bianca Madalina Buisman, Ruschlikon (CH); Quang Anh Duong, San Francisco, CA (US); Anastasia Martynova, Alameda, CA (US); Ayush Jain, Los Altos, CA (US); Yuan Liu, Santa Clara, CA (US); Jonathan David Krause, Mountain View, CA (US); Amit Sanjay Talreja, Santa Clara, CA (US); Rajeev Vijay Rikhye, Fremont, CA (US); Mahvish A. Nagda, Palo Alto, CA (US); Pinal Bavishi, Sunnyvale, CA (US); Christopher James Eicher, Cupertino, CA (US); Abigail Ward, San Mateo, CA (US); Jieming Yu, Jersey City, NJ (US); Louis Wang, San Francisco, CA (US); Dounia Berrada, Saratoga, CA (US); Dale Richard Webster, Redwood City, CA (US); Harshit Kharbanda, Pleasanton, CA (US); Igor Bonaci, Wollerau (CH); Kai Yu, San Francisco, CA (US); Ke Lan, San Jose, CA (US); Kaan Yücer, San Francisco, CA (US); Willa Angel Chen Miller, Sunnyvale, CA (US); Lars Thomas Hansen, Adliswil (CH)

(21) Appl. No.: 18/620,434

(22) Filed: Mar. 28, 2024

Related U.S. Application Data
(60) Provisional application No. 63/494,812, filed on Apr. 7, 2023.

(51) Int. Cl. G16H 50/20; G06T 7/00; G16H 30/40

(52) U.S. Cl. CPC G16H 50/20; G06T 7/0012; G16H 30/40; G06T 2207/20104; G06T 2207/30088

(57) ABSTRACT

Systems and methods for diagnostic visual search can include processing a search query with a plurality of classification models to determine a search query intent and predict potential diagnosis. The search query can include an image that is processed to determine the presence of a body part and may be processed to determine if the search query is descriptive of a diagnostic search query. Based on the intent determination, the image may then be processed by a conditions classification model to determine one or more predicted condition classifications. Condition information can then be obtained and provided based on the one or more predicted condition classifications.

### PRIORITY CLAIM

The present application is based on and claims priority to U.S. Provisional Application No. 63/494,812 having a filing date of Apr. 7, 2023. Applicant claims priority to and the benefit of each of such applications and incorporates all such applications herein by reference in its entirety.

### FIELD

The present disclosure relates generally to diagnostic visual search. More particularly, the present disclosure relates to utilizing a plurality of classification models to determine a search query is requesting a predicted medical diagnosis and processing an image of the search query to determine candidate medical conditions.

### BACKGROUND

Users utilize search engines to find recipes, learn about a television show or movie, determine how to change their oil, and discover new topics and ideas. Additionally, users can utilize web searching to problem solve, whether that includes determining what is wrong with their computer or what is wrong with their skin.

Traditional search techniques can provide a large number of search results. However, some search results may be off topic and/or from untrustworthy sources. Additionally, for image queries, matches may be determined based on portions of the image that may not be relevant to the intent of the user when searching.

### SUMMARY

Aspects and advantages of embodiments of the present disclosure will be set forth in part in the following description, or can be learned from the description, or can be learned through practice of the embodiments.

One example aspect of the present disclosure is directed to a computing system for medical condition visual search. The system can include one or more processors and one or more non-transitory computer-readable media that collectively store instructions that, when executed by the one or more processors, cause the computing system to perform operations. The operations can include obtaining a search query. The search query can include one or more images. The one or more images may depict a body part of a user. The operations can include processing the one or more images with an intent classification model to generate an intent classification. The intent classification can indicate that the search query has a diagnostic search intent. In some implementations, the intent classification model may have been trained to determine a search intent of the user based on one or more features in an input image. The operations can include providing the one or more images to a medical conditions classification model based on the intent classification. The operations can include processing the one or more images with the medical conditions classification model to generate one or more predicted condition classifications. The one or more predicted condition classifications can be descriptive of one or more candidate medical conditions determined to be potentially depicted in the one or more images. The operations can include providing medical condition information associated with the one or more candidate medical conditions.

In some implementations, the operations can include processing the one or more images with a skin classification model to determine the one or more images depict skin. The skin classification model may have been trained to determine whether an input image depicts skin. The operations can include providing the one or more images to the intent classification model based on the one or more images depicting skin. In some implementations, the operations can include obtaining the medical condition information associated with the one or more candidate medical conditions from a curated medical information database. The medical condition information can include a medical condition name and one or more condition images. The one or more condition images can depict an example of the respective candidate medical condition. The one or more condition images can be obtained from a medical condition image database. The medical condition image database can include a plurality of medical condition images selected by one or more medical professionals.

In some implementations, the operations can include processing the one or more images to determine a region of interest and cropping the one or more images to generate one or more cropped images based on the region of interest. The one or more cropped images can be processed with the intent classification model and the medical conditions classification model. The operations can include processing the one or more images to determine a region of interest and generating an annotated image based on the one or more images and the region of interest. The annotated image can include the one or more images with one or more indicators. The one or more indicators can indicate a location of the region of interest in the one or more images. The operations can include providing the annotated image for display with the medical condition information as an output.

In some implementations, providing medical condition information associated with the one or more candidate medical conditions can include providing the medical condition information for display in a search results interface. The search results interface can include a first panel including the medical condition information and a second panel including a plurality of visual search results. The plurality of visual search results can be determined based on a determined visual similarity with the one or more images. In some implementations, the search results interface can include a selectable user interface element. The selectable user interface element can be associated with a particular medical condition of the one or more candidate medical conditions. The selectable user interface element can be provided adjacent to the medical condition information. In some implementations, the operations can include obtaining a selection input associated with the selectable user interface element. The selection input can be descriptive of a selection of the selectable user interface element. The operations can include processing a condition name of the particular medical condition with a search engine to determine a plurality of updated search results associated with the particular medical condition and providing the plurality of updated search results for display.

Another example aspect of the present disclosure is directed to a computer-implemented method skin condition search. The method can include obtaining, by a computing system including one or more processors, a search query. The search query can include one or more images. The method can include processing, by the computing system, the one or more images with a skin classification model to determine the one or more images depict skin. The method can include processing, by the computing system, the one or more images with an intent classification model to generate an intent classification. The intent classification can indicate that the search query has a diagnostic search intent. The intent classification model may have been trained to determine a search intent of the user based on one or more features in an input image. The method can include providing, by the computing system, the one or more images to a dermatology conditions classification model based on the intent classification. The method can include processing, by the computing system, the one or more images with the dermatology conditions classification model to generate one or more predicted condition classifications. The one or more predicted condition classifications can be descriptive of one or more candidate skin conditions. The method can include providing, by the computing system, skin condition information associated with the one or more candidate skin conditions as an output.

In some implementations, the method can include obtaining, by the computing system, the skin condition information from a curated database. The curated database can include a plurality of condition datasets associated with a plurality of different skin conditions. The method can include processing, by the computing system, the one or more images with a search engine to identify a plurality of different visual search results. The plurality of different visual search results can be associated with a plurality of different web resources. The method can include providing, by the computing system, the plurality of different visual search results for display with the skin condition information. The plurality of condition datasets may have been at least one of generated or reviewed by a licensed dermatologist. In some implementations, providing, by the computing system, the plurality of different visual search results for display with the skin condition information can include ordering, by the computing system and via a ranking engine, the plurality of different visual search results based on: a determined visual similarity with the one or more images and a determined topic relevance based on whether the particular visual search result is associated with the one or more candidate skin conditions.

In some implementations, processing, by the computing system, the one or more images with the dermatology conditions classification model to generate the one or more predicted condition classifications can include processing, by the computing system, the one or more images with the dermatology conditions classification model to generate a plurality of predicted condition classifications. Each predicted condition classification can be descriptive of a particular candidate skin condition. Processing, by the computing system, the one or more images with the dermatology conditions classification model to generate the one or more predicted condition classifications can include obtaining, by the computing system, a plurality of skin information datasets associated with the plurality of predicted condition classifications. Each skin information dataset of the plurality of skin information datasets can be associated with a different candidate skin condition. Processing, by the computing system, the one or more images with the dermatology conditions classification model to generate the one or more predicted condition classifications can include providing, by the computing system, the plurality of skin information datasets for display via a carousel interface.

In some implementations, obtaining, by the computing system, the search query can include obtaining, by the computing system, the search query via a user interface of a visual search application. Providing, by the computing system, the skin condition information associated with the one or more candidate skin conditions can include providing, by the computing system, the skin condition information for display via the user interface of the visual search application.

Another example aspect of the present disclosure is directed to one or more non-transitory computer-readable media that collectively store instructions that, when executed by one or more computing devices, cause the one or more computing devices to perform operations. The operations can include obtaining a search query from a user computing system. The search query can include one or more images. In some implementations, the one or more images can depict one or more body parts of a user. The operations can include processing the one or more images with an intent classification model to generate an intent classification. The intent classification can be descriptive of a diagnosis search. The intent classification model may have been trained to determine a search intent of the user based on one or more features in an input image. The operations can include providing the one or more images to a medical condition classification model based on the intent classification. The operations can include processing the one or more images with the medical conditions classification model to generate one or more predicted condition classifications. The one or more predicted condition classifications can be descriptive of one or more candidate medical conditions determined to be potentially depicted in the one or more images. The operations can include obtaining condition information for the one or more candidate medical conditions. The condition information can include one or more example images of the particular candidate medical condition and a condition name. The operations can include processing the one or more images with a search engine to determine one or more visual search results. In some implementations, the one or more visual search results can be determined based on a visual feature similarity with the one or more images. The operations can include providing the one or more visual search results and the condition information to the user computing system.

In some implementations, processing the one or more images with a search engine to determine one or more visual search results can include determining a plurality of candidate visual search results based on the one or more images and determining one or more anatomy visual search results of the plurality of candidate visual search results. The one or more anatomy visual search results can include one or more anatomy images that depict a human body part. The one or more visual search results can include the one or more anatomy visual search results. In some implementations, processing the one or more images with a search engine to determine one or more visual search results can include determining a plurality of candidate visual search results based on the one or more images, determining one or more particular candidate search results of the plurality of candidate visual search results are associated with the one or more candidate medical conditions, and adjusting the ranking of the plurality of candidate visual search results based on determining the one or more particular candidate search results are associated with the one or more candidate medical conditions. In some implementations, the one or more visual search results can be provided in a first panel of a search results interface. The condition information can be provided in a second panel of the search results interface.

Another example aspect of the present disclosure is directed to a computing system for skin condition visual search. The system can include one or more processors and one or more non-transitory computer-readable media that collectively store instructions that, when executed by the one or more processors, cause the computing system to perform operations. The operations can include obtaining a search query. The search query can include one or more images. The one or more images may depict skin of a user. The operations can include processing the one or more images with an intent classification model to generate an intent classification. The intent classification can indicate that the search query has a diagnostic search intent. In some implementations, the intent classification model may have been trained to determine a search intent of the user based on one or more features in an input image. The operations can include providing the one or more images to a dermatology conditions classification model based on the intent classification. The operations can include processing the one or more images with the dermatology conditions classification model to generate one or more predicted condition classifications. The one or more predicted condition classifications can be descriptive of one or more candidate skin conditions determined to be potentially depicted in the one or more images. The operations can include providing skin condition information associated with the one or more candidate skin conditions.

In some implementations, the operations can include processing the one or more images with a skin classification model to determine the one or more images depict skin. The skin classification model may have been trained to determine whether an input image depicts skin. The operations can include providing the one or more images to the intent classification model based on the one or more images depicting skin. In some implementations, the operations can include obtaining the skin condition information associated with the one or more candidate skin conditions from a curated medical information database. The skin condition information can include a skin condition name and one or more condition images. The one or more condition images can depict an example of the respective candidate skin condition. The one or more condition images can be obtained from a medical condition image database. The medical condition image database can include a plurality of skin condition images selected by one or more dermatologists.

In some implementations, the operations can include processing the one or more images to determine a region of interest and cropping the one or more images to generate one or more cropped images based on the region of interest. The one or more cropped images can be processed with the intent classification model and the skin conditions classification model. The operations can include processing the one or more images to determine a region of interest and generating an annotated image based on the one or more images and the region of interest. The annotated image can include the one or more images with one or more indicators. The one or more indicators can indicate a location of the region of interest in the one or more images. The operations can include providing the annotated image for display with the skin condition information as an output.

In some implementations, providing skin condition information associated with the one or more candidate skin conditions can include providing the skin condition information for display in a search results interface. The search results interface can include a first panel including the skin condition information and a second panel including a plurality of visual search results. The plurality of visual search results can be determined based on a determined visual similarity with the one or more images. In some implementations, the search results interface can include a selectable user interface element. The selectable user interface element can be associated with a particular skin condition of the one or more candidate medical conditions. The selectable user interface element can be provided adjacent to the skin condition information. In some implementations, the operations can include obtaining a selection input associated with the selectable user interface element. The selection input can be descriptive of a selection of the selectable user interface element. The operations can include processing a condition name of the particular skin condition with a search engine to determine a plurality of updated search results associated with the particular skin condition and providing the plurality of updated search results for display.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 depicts a block diagram of an example medical condition search system according to example embodiments of the present disclosure.

FIG. 2 depicts a block diagram of an example dermatology visual search system according to example embodiments of the present disclosure.

FIG. 3A depicts a flow chart diagram of an example method to perform skin condition diagnostic search according to example embodiments of the present disclosure.

FIG. 3B depicts a flow chart diagram of an example method to perform medical condition diagnostic search according to example embodiments of the present disclosure.

FIG. 4 depicts a block diagram of an example dermatology visual search flow according to example embodiments of the present disclosure.

FIG. 5 depicts an illustration of an example search interface according to example embodiments of the present disclosure.

FIG. 6 depicts a block diagram of an example search pipeline according to example embodiments of the present disclosure.

FIG. 7 depicts a flow chart diagram of an example method to perform skin condition diagnostic search according to example embodiments of the present disclosure.

FIG. 8 depicts a flow chart diagram of an example method to perform medical condition diagnostic search according to example embodiments of the present disclosure.

FIG. 9 depicts a block diagram of an example search system according to example embodiments of the present disclosure.

FIG. 10A depicts a block diagram of an example computing system that performs diagnostic visual search according to example embodiments of the present disclosure.

FIG. 10B depicts a block diagram of an example computing system that performs diagnostic visual search according to example embodiments of the present disclosure.

Reference numerals that are repeated across plural figures are intended to identify the same features in various implementations.

### DETAILED DESCRIPTION

Generally, the present disclosure is directed to systems and methods for medical condition visual search. In particular, the systems and methods disclosed herein can leverage a plurality of classification models to determine a received search query includes a diagnostic intent and to determine one or more candidate medical conditions that may be associated with the one or more images of the search query. Based on the one or more candidate medical conditions, condition information can be retrieved and provided to the user. Additionally and/or alternatively, the condition information can be provided with one or more visual search results that are responsive to the one or more images of the search query being searched.

Users may be experiencing an abnormality in their health and may decide that searching the symptoms can provide them with insight. For example, a user may be noticing a rash on their skin begin to appear and grow. The appearance of the rash can be worrying and may cause the user to question what is happening. The user may decide to search the internet for a preliminary diagnosis. However, describing the appearance of the rash in words can be difficult and can lead to a wide variety of search results that may not be associated with the same type of ailment. Alternatively and/or additionally, the user may capture an image of the rash and may input the image for searching. However, the search may output search results that are not related to medical conditions (e.g., skin conditions) and may instead be associated with feature similarity with other portions of the image. Additionally, general search results from the web may include inaccurate and/or misleading information, which may cause additional harm to a user.

Medical condition visual search can be implemented in a visual search application (e.g., a viewfinder interface with a search feature and/or a reverse image search interface) to have a dedicated processing flow for visual queries determined to have a medical intent. The medical condition visual search may be provided as part of an augmented-reality interface to provide information about the user's environment in an annotated and immersive format. Alternatively and/or additionally, the medical condition information and/or the other search results may be provided in a search results page interface. The medical condition visual search may be provided in a consumer-facing interface to provide vetted medical information to users lacking medical expertise, which may provide a user with the relevant information to seek the proper medical help, mitigate further issues, and/or better understand potential risks.

Users utilize search engines to find recipes, learn about a television show or movie, determine how to change their oil, and discover new topics and ideas. Additionally, users can utilize web searching to problem solve, whether that includes determining what is wrong with their computer or what is wrong with their skin. Traditional search techniques can provide a large number of search results. However, some search results may be off topic and/or from untrustworthy sources. Additionally, for image queries, matches may be determined based on portions of the image that may not be relevant to the intent of the user when searching.

The systems and methods disclosed herein can provide a dedicated search pipeline for visual searches associated with medical condition queries. The dedicated search pipeline can include determining a visual search query has a medical intent, which can cause the visual search query to be processed with a classifier to generate a classification. The classification can then be utilized for search result filtering and/or ranking and may be utilized to query a vetted medical information database.

The classification models can provide a directed search that can provide information that is directly responsive to the search query without iterative searching. The reduction of searches can reduce the computational resources utilized to identify a specific and accurate set of information. Additionally and/or alternatively, the classification models can be utilized to determine particular databases and condition information to search for, which can reduce the number of databases and resources crawled during search.

Traditional visual search techniques can provide for the obtainment of similar images; however, the similar images may not provide relevant and/or accurate medical information. The medical condition visual search system can ensure that visual search queries with a medical diagnostic intent are responded to with relevant and vetted information. A classification model can be utilized for medical condition classification, which can then be leveraged to tailor the search results to information associated with the classified medical condition.

The systems and methods disclosed herein can leverage intent classification then medical condition classification (e.g., skin condition classification) to identify search queries associated with a request for preliminary diagnosis and provide accurate and vetted information on a predicted medical condition (e.g., a predicted skin condition). For example, a search query including an image may be obtained and processed with an intent classification model to determine an intent classification. If the intent classification is descriptive of a diagnostic query to request information about a potential medical condition, the image of the search query can be provided to a medical condition classification model (e.g., the skin condition classification model). If the intent classification is not descriptive of a diagnostic query to request information about a potential medical condition, the image of the search query may be provided to a general search engine.

The image of the search query can be processed with the medical condition classification model (e.g., a skin condition classification model) to generate and/or determine one or more predicted condition classifications. The one or more predicted condition classifications can be descriptive of one or more candidate medical conditions. In particular, the one or more predicted condition classifications can be descriptive of a preliminary diagnosis of one or more medical conditions. Condition information can then be retrieved and provided to the user based on the one or more predicted condition classifications. The condition information can be associated with the one or more candidate medical conditions. For example, the condition information can be descriptive of a particular medical condition that the image may be descriptive of. In some implementations, the condition information can include a name of the particular medical condition, one or more example images of the particular medical condition (e.g., example images of the particular medical condition as verified by medical professionals), and/or descriptions associated with the particular medical condition (e.g., symptoms, treatments, seriousness, and/or specialists in the user's area).

Additionally and/or alternatively, the image of the search query may be processed with a search engine to determine a plurality of visual search results (e.g., a plurality of images responsive to the image query). The plurality of visual search results can be determined based on a determined visual similarity with the image of the search query. In some implementations, the determination may be based on processing the image with an embedding model to generate a feature embedding. The feature embedding can then be utilized to determine similar feature embeddings associated with candidate search results.

The condition information and the plurality of visual search results can then be provided to a user via a search results interface. The condition information may be in a first panel and the plurality of visual search results may be in a second panel. In some implementations, the plurality of visual search results may be ranked and/or pruned based on the one or more predicted condition classifications. For example, visual search results associated with one or more candidate medical conditions may have their ranking boosted, while other visual search results may be pruned and/or penalized in the ranking of the plurality of visual search results.

The systems and methods disclosed herein can provide utility in preliminary medical diagnostics for a variety of medical fields. Dermatology related searches in particular may be improved based on the systems and methods disclosed herein. Various access restrictions (e.g., shortage of dermatologists and/or cost) can make getting proper medical review of skin conditions difficult. Wait times and cost can hinder the ability of users to get proper and timely medical help. The systems and methods disclosed herein can provide artificial intelligence-based preliminary diagnosis that can mitigate issues associated with a lack of access to a dermatologist. Additionally and/or alternatively, the preliminary diagnosis and the information provided based on the classification outputs can enable a user to be more informed when they do visit a doctor.

The condition classification model may include one or more machine-learned models leveraged to provide differential diagnoses for skin conditions based on images with associated metadata. For example, the machine-learned models can include artificial neural networks. In some implementations, the systems and methods disclosed herein can allow a computing system to receive a plurality of images of a patient's skin. The computing system can use a first processing block (e.g., an embedding model) of a machine-learned skin condition classification model to generate a respective embedding for each of the plurality of images. The computing system can combine the embeddings into a unified image representation associated with the patient's skin and can use a second processing block of the machine-learned skin condition classification model to generate a skin condition classification for the patient's skin based on the unified image representation. In some implementations, the skin condition classification provided by the skin condition classification model can be a differential diagnosis that identifies one or more skin conditions out of a plurality of potential skin conditions. Furthermore, in some implementations, metadata associated with the patient can also be additionally input into the model, and the machine-learned skin condition classification model can be configured to jointly process such additional patient metadata alongside the input imagery to produce the output skin condition classification. For example, the additional patient metadata can include patient demographic information, medical history, and/or other information concerning the patient (e.g., the user).

The systems and methods disclosed herein can be used for diagnostic and educational purposes. As one example usage, a medical professional can, as part of a diagnostic procedure, request a user perform an initial visual search to be input in the user's intake form to provide the medical professional with additional data before the medical visit occurs. The user can capture (e.g., using a camera of a user computing device) a plurality of images of the identified area of the patient's skin using a computing device such as a smartphone or digital camera. The captured images can be provided to a machine-learned skin condition classification model which is located locally and/or remotely. The machine-learned skin condition classification model can generate a skin condition classification for the identified portion of the patient's skin. The skin condition classification can include one or more potential skin conditions and a confidence value associated with each potential skin condition. The medical professional can use the skin condition classification to assist in diagnosing the patient's condition. Thus, the systems and methods can serve as a support role for a medical professional that is treating or examining the patient in person. The use of this system can increase the effectiveness of a medical professional while helping to reduce the time needed to diagnose a patient accurately.

The systems and methods disclosed herein may be optionally provided to the user. For example, a user can be given an option at the time of search and/or in updating their user preferences to opt into medical condition diagnostic visual search. Additionally and/or alternatively, the user may be provided an option to opt-out. The user may control whether the algorithm is utilized on a per use basis, on a per context basis, and/or on a global basis. The medical condition visual search system including the intent classification may be performed in the background for users who opt in such that the user may be provided with a traditional search experience until a diagnostic visual search intent is determined. Additionally and/or alternatively, the users can control whether and how their data is utilized, stored, and/or provided.

In some implementations, a user may be provided with an option to have their image utilized for machine-learned model training, retraining, calibration, and/or parameter tuning. If a user selects the option, the image may be processed to strip identifying features from the image. The identifying feature stripping can include cropping and/or augmenting the image to exclude other objects, tattoos, and/or birthmarks. The image can then be processed by the one or more machine-learned models to generate one or more outputs. One or more medical professionals can then review the image to determine one or more labels (e.g., a skin label (e.g., skin is depicted), a diagnostic label (e.g., the depicted abnormality is atopic dermatitis), and/or an intent label (e.g., the search query is associated with a diagnostic search intent)). The one or more outputs and the one or more labels can be compared to evaluate a loss function, which can be utilized to determine a gradient descent. The gradient descent can be backpropagated to the one or more machine-learned models to adjust one or more parameters.

In some implementations, the image data may be processed with an embedding model locally on a user computing device. The generated embedding can then be transmitted to the server computing system for search. The local embedding generation can reduce the computational cost of transmission, while privately storing the image data on the user computing device. Additionally and/or alternatively, the image data may be processed with an obfuscation model to remove identifying features from the image data before processing with the search engine, the intent classification model, and/or the condition classification model.

In some implementations, the search query including the image data, the intent classification, the condition information, and the search result history may be erased and/or deleted at the completion of each search instance (e.g., once a search application or web page is closed and/or once a non-diagnostic search is received).

The systems and methods of the present disclosure provide a number of technical effects and benefits. As one example, the systems and methods can provide a dedicated search pipeline for visual searches associated with medical condition queries. In particular, the systems and methods disclosed herein can utilize a plurality of classifications models to determine when a search query is associated with a diagnostic intent and can determine candidate medical conditions that may be associated with the query. Specific information for the predicted candidate medical conditions can be obtained and provided to the user. The condition information may be provided with one or more visual search results.

Another technical benefit of the systems and methods of the present disclosure is the ability to leverage predicted condition classifications to provide tailored visual search results. For example, the systems and methods disclosed herein can process an image of a search query with a condition classification model to generate one or more predicted condition classifications. The systems and methods may include leveraging the predicted condition classifications to adjust the search query (e.g., augment the search query to include text data descriptive of the predicted condition classifications), adjust visual search result rankings (e.g., boost the rankings of visual search results associated with the one or more predicted condition classifications), and/or prune the visual search results that are not associated with the one or more predicted condition classifications.

Another example of technical effect and benefit relates to improved computational efficiency and improvements in the functioning of a computing system. For example, the systems and methods disclosed herein can leverage the classification models to provide a directed search that can provide information that is directly responsive to the search query without iterative searching. The reduction of searches can reduce the computational resources utilized to identify a specific and accurate set of information. Additionally and/or alternatively, the classification models can be utilized to determine particular databases and condition information to search for, which can reduce the number of databases and resources crawled during search.

With reference now to the Figures, example embodiments of the present disclosure will be discussed in further detail.

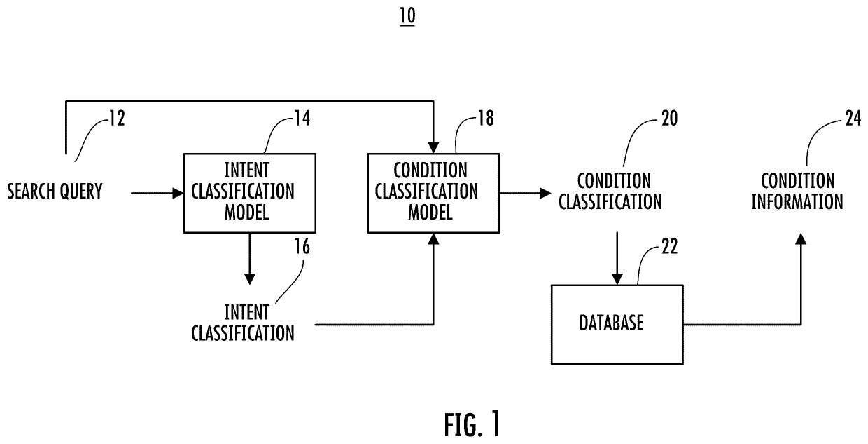

FIG. 1 depicts a block diagram of an example medical condition search system 10 according to example embodiments of the present disclosure. In some implementations, the medical condition search system 10 is configured to receive, and/or obtain, a search query 12 that includes an image descriptive of a body part (e.g., the skin) of a user and, as a result of receipt of the search query 12, generate, determine, and/or provide condition information 24 that describes and/or depicts a predicted candidate medical condition associated with the image 12. Thus, in some implementations, the medical condition search system 10 can include an intent classification model 14 that is operable to determine an intent associated with the input search query and a condition classification model 18 that is operable to determine candidate medical conditions.

In particular, a search query 12 can be obtained from a user computing system. The search query 12 can include an image descriptive of an abnormality on the user's skin. The search query may additionally include text data (e.g., a text query descriptive of a question, symptoms, and/or other details), additional image data, user profile data (e.g., search history data, user browsing history data, and/or other profile information), audio data (e.g., a voice command), and/or latent encoding data.

The search query 12 can be processed with an intent classification model 14 to generate an intent classification 18 associated with the search query 12. The intent classification 14 can be descriptive of a determined intent of the search query 12. For example, the intent classification 18 for the search query 12 may be descriptive of a diagnostic intent based on determining the image depicts an abnormality (e.g., a skin abnormality (e.g., a lesion on the skin). The intent classification model 14 can include one or more machine-learned models and may include one or more classifier heads. The intent classification 16 can be based on semantic understanding, determined objects in the image, determined medical abnormalities in the image, and/or context data. In some implementations, the diagnostic search intent may be determined based on determining the search query 12 is not associated with any other visual search intents or verticals (e.g., shopping and/or natural world understanding). The presence of tattoos, selfies, pornographic imagery, and/or gory/violent content may reduce the likelihood of a diagnostic search intent classification. In some implementations, the intent classification model 14 may be a binary classifier.

Based on the diagnostic intent classification, the image of the search query 12 may then be processed with a condition classification model 18 to generate and/or determine a condition classification 20. The condition classification model 18 can include one or more machine-learned models and may be trained on ground truth images labeled by medical professionals. The condition classification model 18 may include one or more detection models, one or more segmentation models, one or more augmentation models, and/or one or more condition-specific classification models. The condition classification 20 can include a predicted condition classification descriptive of one or more candidate medical conditions. The condition classification 20 can be a preliminary diagnosis of the identified skin abnormality.

The classification models may be trained and/or configured for high specificity (e.g., the classification models may not trigger on an image that does not have a derm intent), low latency, and/or equitable performance (e.g., the classification model may be trained and/or configured to be equitable across different skin types).

Condition information 24 can then be obtained from a database 22 based on the condition classification 20. The condition information 24 can include a medical condition name(s), example medical image(s) for the respective medical condition, and/or description(s) associated with the respective medical condition. The database 22 can include a curated database that includes data verified by medical professionals and may be queried and/or searched based on the condition classification 20.

The condition information 24 can be provided to the user as an output for the search. The condition information 24 may be provided to the user with one or more visual search results.

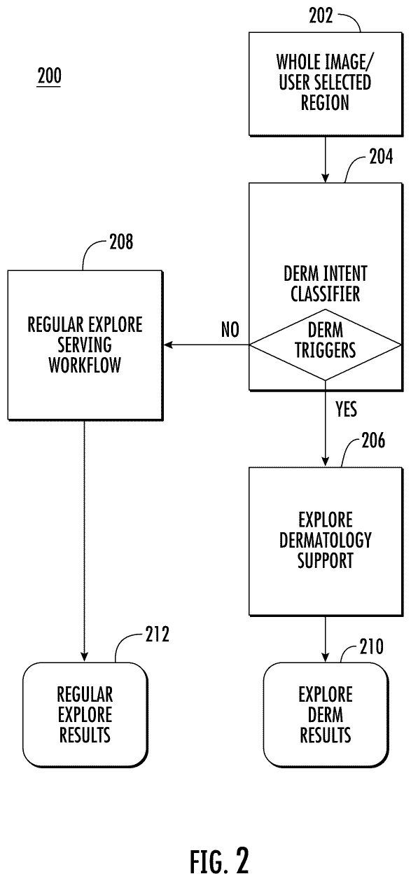

FIG. 2 depicts a block diagram of an example dermatology visual search system 200 according to example embodiments of the present disclosure. The dermatology visual search system 200 is similar to medical condition search system 10 of FIG. 1 except that dermatology visual search system 200 further includes a derm trigger that determines the search path based on the output of the derm intent classifier 204 (i.e., as the intent classification model).

For example, an image 202 can be received for search. The image can be a whole image and/or a portion of an image. The portion can be obtained based on a user selected region and/or based on automatic selection (e.g., based on medical abnormality recognition and/or body part recognition). The image 202 can be processed by a derm intent classifier 204 (i.e., an intent classification model) to determine a search intent for the search query. If the search intent is associated with a dermatology query, the derm trigger may send the image 202 to be processed by a dermatology support system 206. If the search intent is not associated with a dermatology query, the derm trigger may transmit the image 202 to a general search system 208.

Based on a determined dermatology query intent, the image 202 may be processed with the dermatology support system 206 to determine dermatology specific search results 210. The dermatology specific search results 210 can include one or more determined candidate skin conditions associated with a skin abnormality detected in the image 202. The dermatology specific search results 210 can include data curated and/or generated by dermatologists.

In some implementations, a plurality of candidate search results may be determined based on the image 202 (e.g., an embedding based search). The plurality of candidate search results may be reranked and/or filtered based on the one or more determined candidate skin conditions associated with a skin abnormality detected in the image 202. In particular, the search results associated with the one or more determined candidate skin conditions may be more heavily (e.g., favorably) weighted. For example, the ranking can be denoted as:

$${S}_{ir} = {S}_{it}^{{W}_{0}} * {S}_{ptw}^{{W}_{1}} * {S}_{lqp}^{{W}_{2}} * {S}_{localization}^{{W}_{3}}$$

Sᵢₜ can be descriptive of image topicality (e.g., image search result topicality). S_{ptw} can be descriptive of page topicality (e.g., resource topicality). S_{lqp} can be descriptive of landing page quality. S_{localization} can be descriptive of a localization metric. For a dermatology query (or other medical condition query), the landing page quality metric can include Sₚₜ=S_{pt′}*F_{offer_demotion}(is_offer)*F_{page_demotion}(is_relevant_page). The offer demotion can be used to downrank offer results, which is policy-violating, so they are less likely to be selected as part of finalized retrieval response. The demotion function can be denoted as:

$$\begin{matrix}{F}_{offer \_ demotion} ( is\_offer ) = 0 if referrer is offer \\ = 1 otherwise\end{matrix} .$$

The page demotion can be denoted as:

$$\begin{matrix}{F}_{page \_ demotion} ( is\_relevant \_page ) = 0 if referrer has matched \\ page category \\ = 1 otherwise\end{matrix} .$$

The general search system 208 may process the image 208 to determine one or more general search results 212. The general search results 212 can include images, links, and/or text obtained from web resources across the internet.

In some implementations, the image 202 be processed with both the dermatology support system 206 and the general search system 208 to provide a search results page that includes both dermatology specific data and general data.

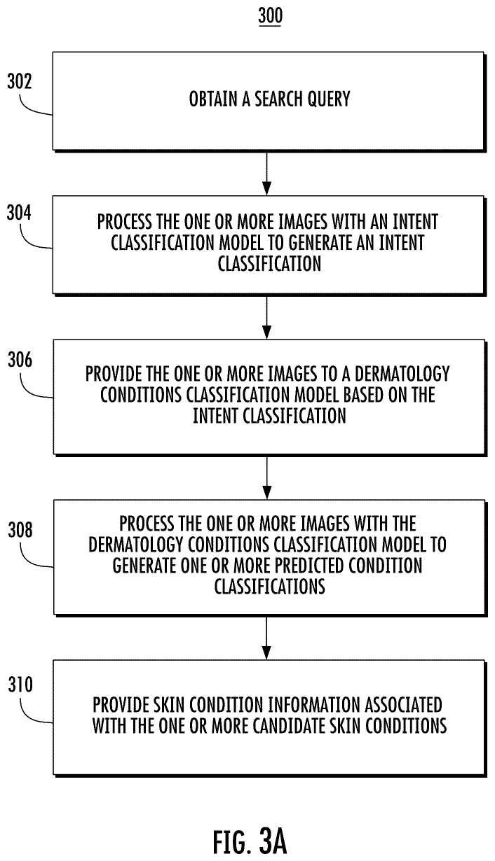

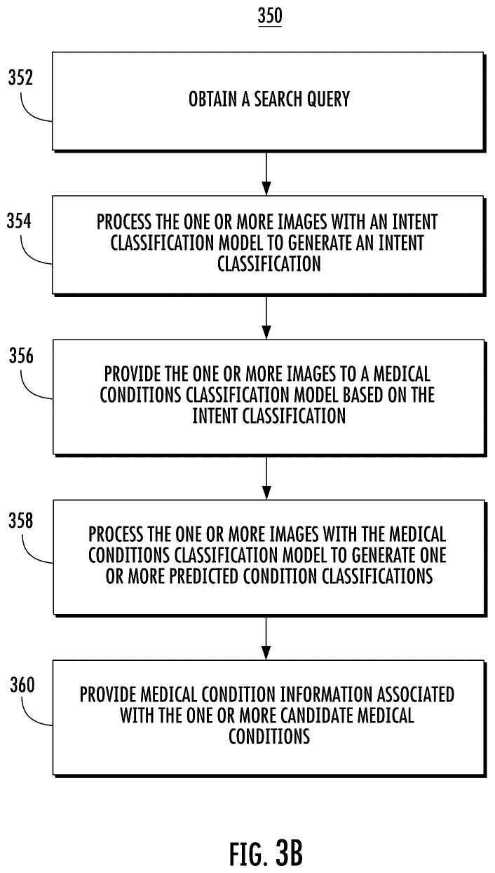

FIG. 3A depicts a flow chart diagram of an example method to perform according to example embodiments of the present disclosure. Although FIG. 3A depicts steps performed in a particular order for purposes of illustration and discussion, the methods of the present disclosure are not limited to the particularly illustrated order or arrangement. The various steps of the method 300 can be omitted, rearranged, combined, and/or adapted in various ways without deviating from the scope of the present disclosure.

At 302, a computing system can obtain a search query. The search query can include one or more images. The one or more images can depict skin of a user. The one or more images can be input by a user computing system. In some implementations, the one or more images can be obtained from a database (e.g., an image gallery of a user). Alternatively and/or additionally, the one or more images may be captured by a user computing device.

At 304, the computing system can process the one or more images with an intent classification model to generate an intent classification. The intent classification can indicate that the search query has a diagnostic search intent. The intent classification model may have been trained to determine a search intent of the user based on one or more features in an input image.

In some implementations, the computing system can process the one or more images with a skin classification model to determine the one or more images depict skin. The skin classification model may have been trained to determine whether an input image depicts skin. The one or more images can be provided to the intent classification model based on the one or more images depicting skin.

At 306, the computing system can provide the one or more images to a dermatology conditions classification model based on the intent classification.

At 308, the computing system can process the one or more images with the dermatology conditions classification model to generate one or more predicted condition classifications. The one or more predicted condition classifications can be descriptive of one or more candidate skin conditions determined to be potentially depicted in the one or more images.

At 310, the computing system can provide skin condition information associated with the one or more candidate skin conditions. Providing skin condition information associated with the one or more candidate skin conditions can include providing the skin condition information for display in a search results interface. The search results interface can include a first panel that includes the skin condition information and a second panel that includes a plurality of visual search results. The plurality of visual search results can be determined based on a determined visual similarity with the one or more images.

In some implementations, the search results interface can include a selectable user interface element. The selectable user interface element can be associated with a particular skin condition of the one or more candidate skin conditions. In some implementations, the selectable user interface element can be provided adjacent to the skin condition information. Additionally and/or alternatively, the computing system can obtain a selection input associated with the selectable user interface element. The selection input can be descriptive of a selection of the selectable user interface element. The computing system can process a condition name of the particular skin condition with a search engine to determine a plurality of updated search results associated with the particular skin condition and can provide the plurality of updated search results for display.

In some implementations, the computing system can obtain skin condition information associated with the one or more candidate skin conditions. The skin condition information can include a skin condition name and one or more condition images. The one or more condition images can depict an example of the respective candidate skin condition. The one or more condition images may be obtained from a skin condition image database. Additionally and/or alternatively, the skin condition image database can include a plurality of skin condition images selected by one or more dermatologists.

In some implementations, the computing system can process the one or more images to determine a region of interest and can crop the one or more images to generate one or more cropped images based on the region of interest. The one or more cropped images can then be processed with the intent classification model and the dermatology conditions classification model.

Additionally and/or alternatively, the computing system can process the one or more images to determine a region of interest and can generate an annotated image based on the one or more images and the region of interest. The annotated image can include the one or more images with one or more indicators. The one or more indicators can indicate a location of the region of interest in the one or more images. The annotated image can be provided for display with the skin condition information as an output.

In some implementations, the systems and methods disclosed herein can be leveraged for other medical condition visual searches outside of just skin condition visual search. For example, the systems and methods can be utilized for the fields of hair loss, nail conditions, infections (e.g., abscess, cellulitis, and/or HSV), cardiovascular (e.g., arterial insufficiency, vasculitis, and/or venous ulcers), dental/oral/ENT issues (e.g., aphthous ulcers and/or angular cheilitis), podiatry (e.g., corns and/or foot lesions), genetic issues (e.g., neurofibromas and/or xeroderma), rheumatology (e.g., erythema nodosum and/or dermatomyositis), diabetes risk (e.g., acanthosis nigricans), hematology, oncology (e.g., T-cell lymphoma and/or Kaposi's sarcoma), GI (e.g., pyoderma gangrenosum), trauma (e.g., burns, abrasions, and/or wounds), cosmetic (e.g., freckles and/or benign lesions), and/or eye conditions.

FIG. 3B depicts a flow chart diagram of an example method to perform according to example embodiments of the present disclosure. Although FIG. 3B depicts steps performed in a particular order for purposes of illustration and discussion, the methods of the present disclosure are not limited to the particularly illustrated order or arrangement. The various steps of the method 350 can be omitted, rearranged, combined, and/or adapted in various ways without deviating from the scope of the present disclosure.

At 352, a computing system can obtain a search query. The search query can include one or more images. The one or more images depict a body part of a user. For example, the one or more images may depict skin, teeth, eyes, tongue, feet, hands, cars, and/or other body parts. In some implementations, the search query may include a multimodal query. For example, the search query may include the one or more images and a text query associated with the one or more images (e.g., one or more images of a rash and a text query stating “why do I have this rash?”). In some implementations, the multimodal query may include text data and/or audio data descriptive of a context for the one or more images, which may include a list of symptoms, a location of the user, when the ailment occurred, a family history, and/or other relevant context information).

At 354, the computing system can process the one or more images with an intent classification model to generate an intent classification. The intent classification can indicate that the search query has a diagnostic search intent. The intent classification model may have been trained to determine a search intent of the user based on one or more features in an input image. In some implementations, the intent classification model may process context data, audio data, and/or text data with the image data to generate the intent classification. The context data can include a search history, a browsing history, a user location, and/or other context data. The audio data and/or the text data may be descriptive of user inputs associated with a question, a command, a set of symptoms, and/or other information. The intent classification may be determined based on determining an abnormality in the image. In some implementations, a body part may be identified as healthy, which may cause the intent classification model to determine the search query does not include a diagnostic search intent. Additionally, and/or alternatively, a diagnostic search intent may be determined based at least in part to determining a medical abnormality is depicted. Text data descriptive of symptoms, a question about whether something is healthy, previous search queries and/or web page visits associated with medical intent, and/or other additional information may be utilized in determining the diagnostic search intent.

In some implementations, the computing system can process the one or more images with a body part classification model (e.g., a skin classification model, a dental classification model, etc.) to determine the one or more images depict a particular body part (e.g., skin, teeth, foot, eyes, etc.). The body part classification model may have been trained to determine whether an input image depicts body parts. The computing system can provide the one or more images to the intent classification model based on determining the one or more images depicting the particular body part.

At 356, the computing system can provide the one or more images to a medical conditions classification model based on the intent classification. The one or more images may be provided to the medical conditions classification model via an application programming interface. In some implementations, a particular medical conditions classification model of a plurality of candidate medical conditions classification model based on the intent classification. For example, the intent classification may include data descriptive of a particular field of medical information being queried (e.g., dermatology, podiatry, ophthalmology, etc.). A particular medical conditions classification model associated with that particular field may then be determined and utilized. Alternatively and/or additionally, the medical conditions classification model may be trained and/or configured to classify conditions in a plurality of different medical fields.

In some implementations, the computing system can process the one or more images to determine a region of interest. The computing system can then crop the one or more images to generate one or more cropped images based on the region of interest. The one or more cropped images can then be processed with the intent classification model and the medical conditions classification model. In some implementations, the region of interest may be determined based at least in part on a user input selecting at least a portion of the one or more images. The region of interest may be determined by processing the one or more images with a machine-learned detection model. The cropping may be performed with a machine-learned segmentation model. The segmentation model may perform the segmentation based on bounding boxes generated by the detection model.

At 358, the computing system can process the one or more images with the medical conditions classification model to generate one or more predicted condition classifications. The one or more predicted condition classifications can be descriptive of one or more candidate medical conditions determined to be potentially depicted in the one or more images. The medical conditions classification model may be trained and/or configured for multimodal processing. In some implementations, the medical conditions classification model can process a multimodal query and/or context data to generate the one or more predicted condition classifications. For example, the one or more predicted condition classifications may be determined based on image feature classification and symptoms described in the text data of the multimodal query. The one or more candidate medical conditions can include skin conditions, hair conditions, infections, cardiovascular conditions, dental conditions, oral conditions, podiatry conditions, genetic conditions, rheumatology conditions, diabetes side effects, blood conditions, cancer classifications, gastrointestinal conditions, trauma conditions, benign cosmetic conditions, eye conditions, and/or other medical conditions.

At 360, the computing system can provide medical condition information associated with the one or more candidate medical conditions. Providing medical condition information associated with the one or more candidate medical conditions can include providing the medical condition information for display in a search results interface. In some implementations, the search results interface can include a first panel including the medical condition information and a second panel including a plurality of visual search results. The plurality of visual search results can be determined based on a determined visual similarity with the one or more images. The search results may be filtered and/or ranked based on the one or more predicted condition classifications (e.g., search results associated with the one or more candidate medical conditions may be weighted more heavily in rankings and/or search results that are not associated with the one or more candidate medical conditions may be pruned (or filtered out)).

In some implementations, the search results interface can include a selectable user interface element. The selectable user interface element can be associated with a particular medical condition of the one or more candidate medical conditions. The selectable user interface element can be provided adjacent to the medical condition information. In some implementations, the computing system can obtain a selection input associated with the selectable user interface element. The selection input can be descriptive of a selection of the selectable user interface element. The computing system may then process a condition name of the particular skin condition with a search engine to determine a plurality of updated search results associated with the particular medical condition and provide the plurality of updated search results for display.

In some implementations, the computing system can obtain the medical condition information associated with the one or more candidate medical conditions from a curated medical information database. The medical condition information can include a medical condition name and/or one or more condition images. The one or more condition images can depict an example of the respective candidate medical condition. In some implementations, the one or more condition images can be obtained from a medical condition image database. The medical condition image database can include a plurality of medical condition images selected by one or more medical professionals.

In some implementations, the computing system can process the one or more images to determine a region of interest and generate an annotated image based on the one or more images and the region of interest. The annotated image can include the one or more images with one or more indicators. The one or more indicators may indicate a location of the region of interest in the one or more images. The computing system can then provide the annotated image for display with the medical condition information as an output. The annotated image may include annotations generated based on the medical condition information (e.g., label overlays, treatment animations, etc.).

Additionally and/or alternatively, the computing system may leverage one or more generative models to generate an understandable, direct, and/or detailed output for the user. In particular, the computing system can process the one or more images, text data, context data, the one or more predicted condition classifications, and/or the medical condition information with a generative model (e.g., a large language model (e.g., an autoregressive language model), a vision language model, an image generation model (e.g., a diffusion model), and/or other generative models) to generate a model-generated output. The computing system can then provide the model-generated output to the user. The model-generated output can include a natural language output and/or a multimodal output that includes a plain language explanation of the determined one or more predicted condition classifications and/or the medical condition information. In some implementations, the model-generated output can include a natural language response to the text query (e.g., the question) in the multimodal query. The natural language response can include the medical information while being formatted to be conversationally responsive to the text query.

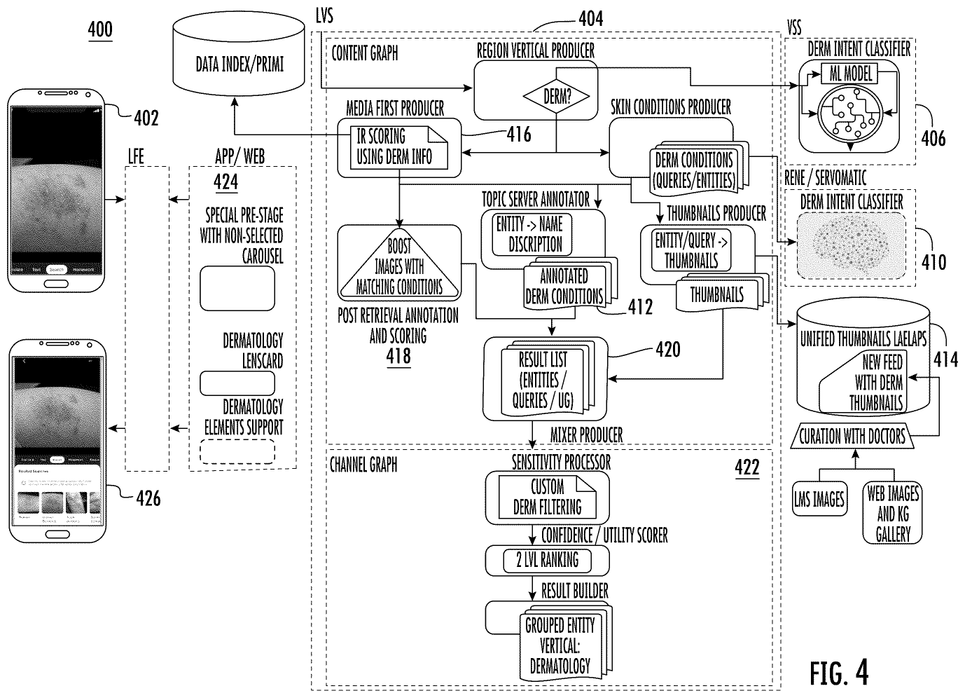

FIG. 4 depicts a block diagram of an example dermatology visual search flow 400 according to example embodiments of the present disclosure. In particular, image data 402 can be obtained from a user computing system. The image 402 can be processed with a skin classification model 404 to determine whether the image 402 depicts skin. If the skin classification model 404 determines the image 402 depicts skin, the image 406 can then be processed with an intent classification model 406 to generate an intent classification. If the intent classification is descriptive of a diagnostic intent (e.g., an intent to receive a preliminary diagnosis for the skin abnormality), the image 402 may then be processed with a skin condition classification model 410 to determine a preliminary diagnosis of predicted candidate medical conditions.

Skin condition information 412 can then be obtained based on the preliminary diagnosis. The skin condition information 412 can include annotated dermatology condition data and/or example image thumbnails for each of the predicted candidate medical conditions. The skin condition information 412 including the thumbnails may be obtained from a curated medical condition database 414. The curated medical condition database 414 can include vetted information and/or images that were curated (e.g., selected and/or input) by medical professionals (e.g., doctors (e.g., dermatologists)).

Additionally and/or alternatively, the image data 402 can be processed with a search engine 416. In some implementations, the search engine 416 can process the image data 402 with context data and/or data descriptive of the preliminary diagnosis. The search engine 416 can determine a plurality of search results based at least in part on features in the image data 402. The ranking for the plurality of search results may be adjusted based on a post retrieval annotation and scoring block 418, which may include a ranking engine that adjusts the ranking based on a determined association with dermatology and/or one or more predicted candidate medical conditions of the preliminary diagnosis.

The skin condition information 412 and the plurality of search results can then be provided to the user via a results list 420. The results list 420 may be ordered 422 and/or ranked based on a level of sensitivity, topicality, a level of confidence, and/or the vertical. The results list 420 may be provided for display via one or more interfaces 424, which may include a search results interface, a viewfinder interface, and/or a dedicated diagnosis interface. The one or more interfaces 424 can include a search result carousel, a graphical card interface for displaying the image 402 with the medical information, a viewfinder interface panel with a search results carousel panel, and/or other interface features. The results list 420 may be transmitted to a user computing device 426 for display.

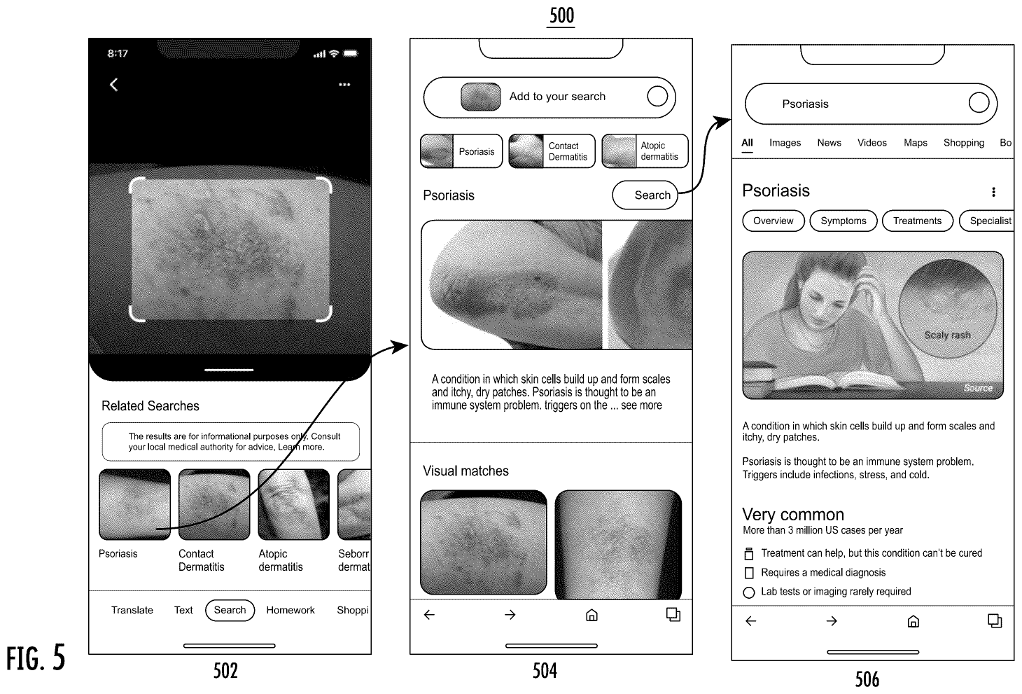

FIG. 5 depicts an illustration of an example search interface 500 according to example embodiments of the present disclosure. In particular, the search interface 500 depicted includes an initial interface 502 that can depict the input image and a set of candidate skin conditions. The initial interface 502 may determine a region of interest and indicate the region of interest by annotating the image with a bounding box. Additionally, the initial interface 502 can include a thumbnail and a caption for each candidate skin condition of the set of candidate skin conditions determined to be candidate diagnosis for the skin abnormality.

A user may select a particular candidate skin condition tile, which may cause a visual search results interface 504 to be displayed. The visual search results interface 504 can include skin condition information for the selected particular candidate skin condition. The skin condition information can include example images, a condition name, and a condition description. The visual search results interface 504 may include a plurality of visual search results descriptive of images that are determined to be visually similar to the input image. In some implementations, the user may be provided with search suggestions based on the output of a transformer model that may be trained on image and text data to provide suggestions for providing a more accurate preliminary diagnosis and/or search. The search suggestions can include inputting a particular image associated with the skin condition, inputting symptoms via text input, and/or answering one or more questions.

A user may then select a search icon that searches the name of the particular skin condition to provide a condition search results interface 506 that provides more detailed information on the selected particular skin condition.

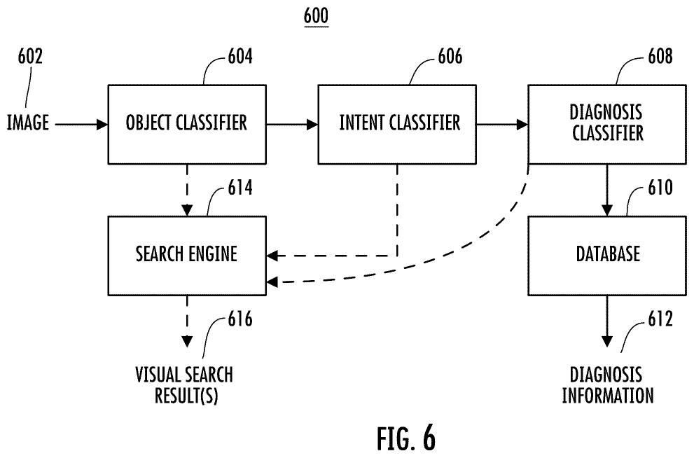

FIG. 6 depicts a block diagram of an example search pipeline 600 according to example embodiments of the present disclosure. In particular, an image 602 can be obtained and processed with an object classifier 604 to determine whether the image 602 depicts a body part. If the image 602 does not depict a body part, then the image 602 may be provided to a search engine 614 for general search. If the image 602 is determined to depict a body part, then the image 602 is processed with an intent classifier 606 to determine an intent for the image 602. If the intent is a general search intent, then the image 602 may be transmitted to the search engine 614. If the intent is determined to be a diagnostic intent, then the image 602 may be processed with a diagnosis classifier 608 that can output a determined preliminary diagnosis for the abnormality depicted in the image 602.

The preliminary diagnosis can then be utilized to query a curated medical database 610 to determine diagnosis information 612 to provide to the user based on the preliminary diagnosis. The diagnosis information 612 can include condition name(s), example image(s), and/or description data. The object classifier 604, the intent classifier 606, and the diagnosis classifier 608 may be machine-learned models trained on labeled training datasets.

In some implementations, the preliminary diagnosis and the image 602 may be processed with the search engine 614 to determine a plurality of visual search results 616 to provide with the diagnosis information 612.

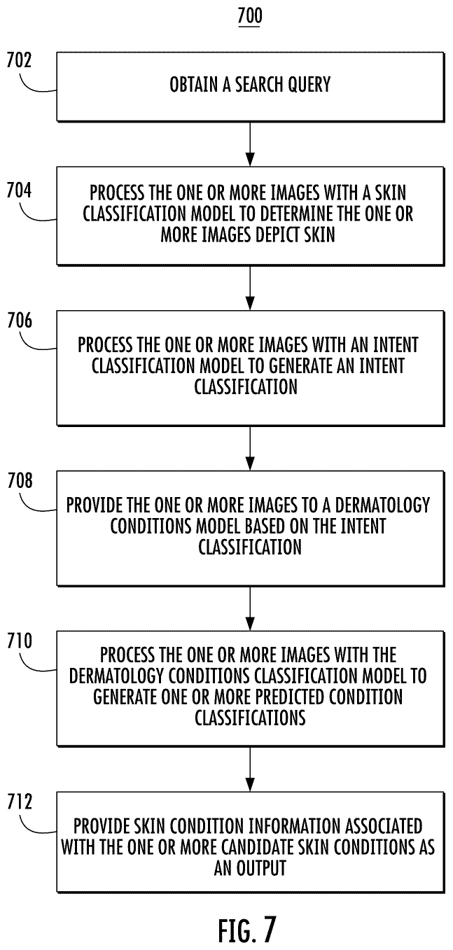

FIG. 7 depicts a flow chart diagram of an example method to perform according to example embodiments of the present disclosure. Although FIG. 7 depicts steps performed in a particular order for purposes of illustration and discussion, the methods of the present disclosure are not limited to the particularly illustrated order or arrangement. The various steps of the method 700 can be omitted, rearranged, combined, and/or adapted in various ways without deviating from the scope of the present disclosure.

At 702, a computing system can obtain a search query. The search query can include one or more images. Obtaining the search query can include obtaining the search query via a user interface of a visual search application. The search query can be a visual query. In some implementations, the search query can include a multimodal query. The multimodal query may include an image and text asking a question about the image. Alternatively and/or additionally, the text may be descriptive of symptoms that provide additional context for the image.

At 704, the computing system can process the one or more images with a skin classification model to determine the one or more images depict skin. The skin classification model may be trained on a diverse image dataset associated with a plurality of different skin types.

At 706, the computing system can process the one or more images with an intent classification model to generate an intent classification. The intent classification can indicate that the search query has a diagnostic search intent. In some implementations, the intent classification model may have been trained to determine a search intent of the user based on one or more features in an input image. The intent classification model processing may be performed in response to determining the one or more images depict skin.

At 708, the computing system can provide the one or more images to a dermatology conditions classification model based on the intent classification. The one or more images may be provided via one or more application programming interfaces. The one or more images may be provided to the dermatology conditions classification model in response to determining the search query has a diagnostic search intent.

At 710, the computing system can process the one or more images with the dermatology conditions classification model to generate one or more predicted condition classifications. The one or more predicted condition classifications can be descriptive of one or more candidate skin conditions.

In some implementations, processing the one or more images with the dermatology conditions classification model to generate the one or more predicted condition classifications can include processing the one or more images with the dermatology conditions classification model to generate a plurality of predicted condition classifications. Each predicted condition classification can be descriptive of a particular candidate skin condition. A plurality of skin information datasets associated with the plurality of predicted condition classifications can then be obtained. Each skin information dataset of the plurality of skin information datasets can be associated with a different candidate skin condition. The plurality of skin information datasets can be provided for display via a carousel interface.

At 712, the computing system can provide skin condition information associated with the one or more candidate skin conditions as an output. Providing the skin condition information associated with the one or more candidate skin conditions can include providing the skin condition information for display via the user interface of the visual search application.

In some implementations, the computing system can obtain the skin condition information from a curated database. The curated database can include a plurality of condition datasets associated with a plurality of different skin conditions. The plurality of condition datasets may have been at least one of generated or reviewed by a licensed dermatologist. The computing system can process the one or more images with a search engine to identify a plurality of different visual search results. The plurality of different visual search results can be associated with a plurality of different web resources. The plurality of different visual search results can be provided for display with the skin condition information.

Additionally and/or alternatively, providing the plurality of different visual search results for display with the skin condition information can include ordering, via a ranking engine, the plurality of different visual search results based on a determined visual similarity with the one or more images and a determined topic relevance based on whether the particular visual search result is associated with the one or more candidate skin conditions.

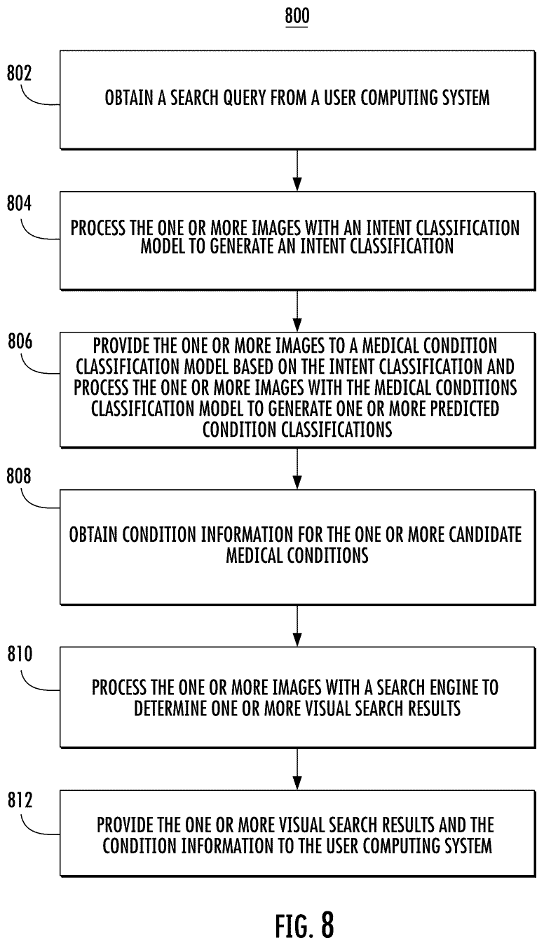

FIG. 8 depicts a flow chart diagram of an example method to perform according to example embodiments of the present disclosure. Although FIG. 8 depicts steps performed in a particular order for purposes of illustration and discussion, the methods of the present disclosure are not limited to the particularly illustrated order or arrangement. The various steps of the method 800 can be omitted, rearranged, combined, and/or adapted in various ways without deviating from the scope of the present disclosure.

At 802, a computing system can obtain a search query from a user computing system. The search query can include one or more images. The one or more images can depict one or more body parts of a user.

At 804, the computing system can process the one or more images with an intent classification model to generate an intent classification. The intent classification can indicate that the search query has a diagnostic search intent. In some implementations, the intent classification model may have been trained to determine a search intent of the user based on one or more features in an input image.

At 806, the computing system can provide the one or more images to a medical condition classification model based on the intent classification and process. The one or more images can be provided to the medical condition classification model in response to determining the search query has a diagnostic search intent.

At 808, the computing system can process the one or more images with the medical conditions classification model to generate one or more predicted condition classifications. The one or more predicted condition classifications can be descriptive of one or more candidate medical conditions determined to be potentially depicted in the one or more images.

At 810, the computing system can obtain condition information for the one or more candidate medical conditions. The condition information can include one or more example images of the particular candidate medical condition and a condition name.

At 812, the computing system can process the one or more images with a search engine to determine one or more visual search results. The one or more visual search results can be determined based on a visual feature similarity with the one or more images.

In some implementations, processing the one or more images with a search engine to determine one or more visual search results can include determining a plurality of candidate visual search results based on the one or more images and determining one or more anatomy visual search results of the plurality of candidate visual search results. The one or more anatomy visual search results can include one or more anatomy images that depict a human body part. The one or more visual search results can include the one or more anatomy visual search results.

Alternatively and/or additionally, processing the one or more images with a search engine to determine one or more visual search results can include determining a plurality of candidate visual search results based on the one or more images, determining one or more particular candidate search results of the plurality of candidate visual search results are associated with the one or more candidate medical conditions, and adjusting the ranking of the plurality of candidate visual search results based on determining the one or more particular candidate search results are associated with the one or more candidate medical conditions.

The computing system can provide the one or more visual search results and the condition information to the user computing system. The one or more visual search results can be provided in a first panel of a search results interface. The condition information can be provided in a second panel of the search results interface.

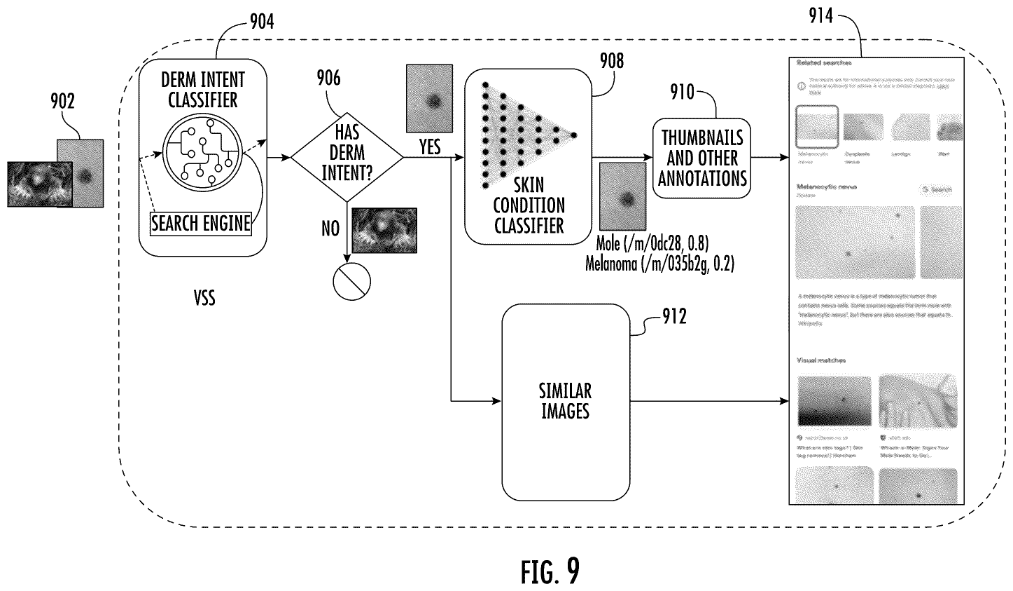

FIG. 9 depicts a block diagram of an example search system 900 according to example embodiments of the present disclosure. In particular, the search system 900 can process image data 902 and can determine whether a dermatology specific search is to be performed and/or if a general search is to be performed.

For example, image data 902 can be processed with an intent classification model 904 to determine an intent classification associated with the search query. Based on the intent classification, a dermatology trigger 906 can determine whether the dermatology specific is to be performed. In particular, a diagnostic search intent may trigger the dermatology specific search, while a shopping intent may trigger a general search.

The dermatology specific search can include processing the image data 902 with a skin condition classification model 908 to determine a preliminary diagnosis for the skin abnormality depicted in the image data 902. Skin condition information 910 can then be obtained based on the preliminary diagnosis. Additionally and/or alternatively, the image data 902 may be processed to determine similar images 912.

The skin condition information 910 and the similar images 912 can then be provided for display via a search results interface 914. The search results interface 914 can include a plurality of panels associated with different types of search results. The plurality of different panels may include a generative model response panel that depicts a generative model response to the search query. For example, the search query, the skin condition information, search results, and/or other details may be processed with a generative model (e.g., a generative language model) to generate a model-generated response that can be provided for display in the search results interface 914. Additionally, and/or alternatively, the search results interface 914 may include a doctor curated information panel, an annotated images panel, a web resources panel, and/or one or more other panels.

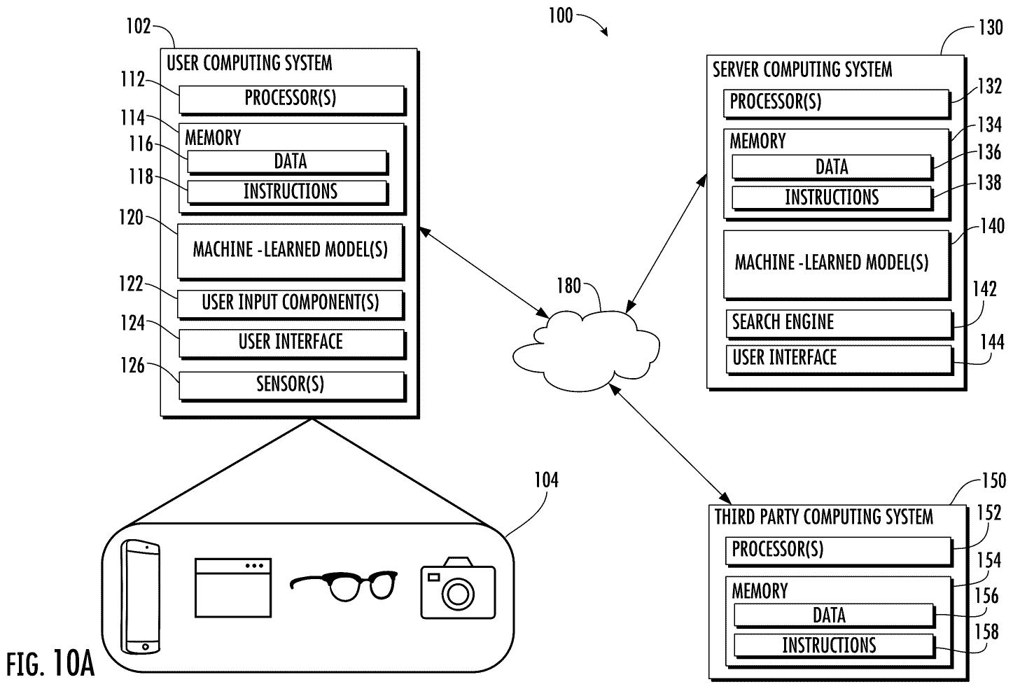

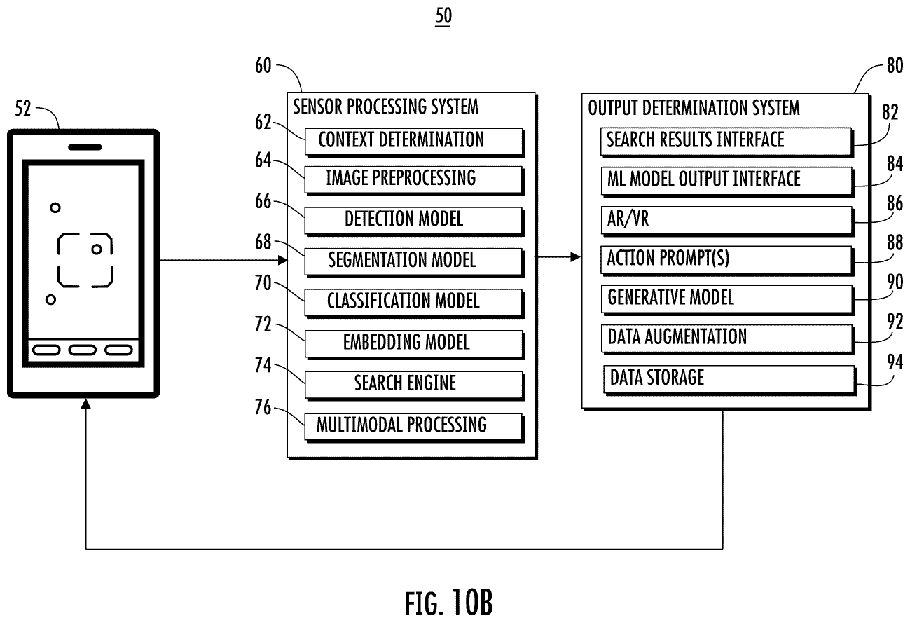

FIG. 10A depicts a block diagram of an example computing system 100 that performs diagnostic visual search according to example embodiments of the present disclosure. The system 100 includes a user computing system 102, a server computing system 130, and/or a third computing system 150 that are communicatively coupled over a network 180.

The user computing system 102 can include any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing system 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the user computing system 102 to perform operations.

In some implementations, the user computing system 102 can store or include one or more machine-learned models 120. For example, the machine-learned models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks.

In some implementations, the one or more machine-learned models 120 can be received from the server computing system 130 over network 180, stored in the user computing device memory 114, and then used or otherwise implemented by the one or more processors 112. In some implementations, the user computing system 102 can implement multiple parallel instances of a single machine-learned model 120 (e.g., to perform parallel machine-learned model processing across multiple instances of input data and/or detected features).

More particularly, the one or more machine-learned models 120 may include one or more detection models, one or more classification models, one or more segmentation models, one or more augmentation models, one or more generative models, one or more natural language processing models, one or more optical character recognition models, and/or one or more other machine-learned models. The one or more machine-learned models 120 can include one or more transformer models. The one or more machine-learned models 120 may include one or more neural radiance field models, one or more diffusion models, and/or one or more autoregressive language models.

The one or more machine-learned models 120 may be utilized to detect one or more object features. The detected object features may be classified and/or embedded. The classification and/or the embedding may then be utilized to perform a search to determine one or more search results. Alternatively and/or additionally, the one or more detected features may be utilized to determine an indicator (e.g., a user interface element that indicates a detected feature) is to be provided to indicate a feature has been detected. The user may then select the indicator to cause a feature classification, embedding, and/or search to be performed. In some implementations, the classification, the embedding, and/or the searching can be performed before the indicator is selected.

In some implementations, the one or more machine-learned models 120 can process image data, text data, audio data, and/or latent encoding data to generate output data that can include image data, text data, audio data, and/or latent encoding data. The one or more machine-learned models 120 may perform optical character recognition, natural language processing, image classification, object classification, text classification, audio classification, context determination, action prediction, image correction, image augmentation, text augmentation, sentiment analysis, object detection, error detection, inpainting, video stabilization, audio correction, audio augmentation, and/or data segmentation (e.g., mask based segmentation).

Machine-learned model(s) can be or include one or multiple machine-learned models or model components. Example machine-learned models can include neural networks (e.g., deep neural networks). Example machine-learned models can include non-linear models or linear models. Example machine-learned models can use other architectures in lieu of or in addition to neural networks. Example machine-learned models can include decision tree based models, support vector machines, hidden Markov models, Bayesian networks, linear regression models, k-means clustering models, etc.

Example neural networks can include feed-forward neural networks, recurrent neural networks (RNNs), including long short-term memory (LSTM) based recurrent neural networks, convolutional neural networks (CNNs), diffusion models, generative-adversarial networks, or other forms of neural networks. Example neural networks can be deep neural networks. Some example machine-learned models can leverage an attention mechanism such as self-attention. For example, some example machine-learned models can include multi-headed self-attention models.

Machine-learned model(s) can include a single or multiple instances of the same model configured to operate on data from input(s). Machine-learned model(s) can include an ensemble of different models that can cooperatively interact to process data from input(s). For example, machine-learned model(s) can employ a mixture-of-experts structure. See, e.g., Zhou et al., Mixture-of-Experts with Expert Choice Routing, ARXIV: 2202.09368v2 (Oct. 14, 2022).

Input(s) can generally include or otherwise represent various types of data. Input(s) can include one type or many different types of data. Output(s) can be data of the same type(s) or of different types of data as compared to input(s). Output(s) can include one type or many different types of data.

Example data types for input(s) or output(s) include natural language text data, software code data (e.g., source code, object code, machine code, or any other form of computer-readable instructions or programming languages), machine code data (e.g., binary code, assembly code, or other forms of machine-readable instructions that can be executed directly by a computer's central processing unit), assembly code data (e.g., low-level programming languages that use symbolic representations of machine code instructions to program a processing unit), genetic data or other chemical or biochemical data, image data, audio data, audiovisual data, haptic data, biometric data, medical data, financial data, statistical data, geographical data, astronomical data, historical data, sensor data generally (e.g., digital or analog values, such as voltage or other absolute or relative level measurement values from a real or artificial input, such as from an audio sensor, light sensor, displacement sensor, etc.), and the like. Data can be raw or processed and can be in any format or schema.

In multimodal inputs or outputs, example combinations of data types include image data and audio data, image data and natural language data, natural language data and software code data, image data and biometric data, sensor data and medical data, etc. It is to be understood that any combination of data types in an input or an output can be present.

An example input can include one or multiple data types, such as the example data types noted above. An example output can include one or multiple data types, such as the example data types noted above. The data type(s) of input can be the same as or different from the data type(s) of output. It is to be understood that the example data types noted above are provided for illustrative purposes only. Data types contemplated within the scope of the present disclosure are not limited to those examples noted above.

Additionally or alternatively, one or more machine-learned models 140 can be included in or otherwise stored and implemented by the server computing system 130 that communicates with the user computing system 102 according to a client-server relationship. For example, the machine-learned models 140 can be implemented by the server computing system 140 as a portion of a web service (e.g., a viewfinder service, a visual search service, an image processing service, an ambient computing service, and/or an overlay application service). Thus, one or more models 120 can be stored and implemented at the user computing system 102 and/or one or more models 140 can be stored and implemented at the server computing system 130.

The user computing system 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

In some implementations, the user computing system can store and/or provide one or more user interfaces 124, which may be associated with one or more applications. The one or more user interfaces 124 can be configured to receive inputs and/or provide data for display (e.g., image data, text data, audio data, one or more user interface elements, an augmented-reality experience, a virtual reality experience, and/or other data for display. The user interfaces 124 may be associated with one or more other computing systems (e.g., server computing system 130 and/or third party computing system 150). The user interfaces 124 can include a viewfinder interface, a search interface, a generative model interface, a social media interface, and/or a media content gallery interface.

The user computing system 102 may include and/or receive data from one or more sensors 126. The one or more sensors 126 may be housed in a housing component that houses the one or more processors 112, the memory 114, and/or one or more hardware components, which may store, and/or cause to perform, one or more software packets. The one or more sensors 126 can include one or more image sensors (e.g., a camera), one or more lidar sensors, one or more audio sensors (e.g., a microphone), one or more inertial sensors (e.g., inertial measurement unit), one or more biological sensors (e.g., a heart rate sensor, a pulse sensor, a retinal sensor, and/or a fingerprint sensor), one or more infrared sensors, one or more location sensors (e.g., GPS), one or more touch sensors (e.g., a conductive touch sensor and/or a mechanical touch sensor), and/or one or more other sensors. The one or more sensors can be utilized to obtain data associated with a user's environment (e.g., an image of a user's environment, a recording of the environment, and/or the location of the user).

The user computing system 102 may include, and/or be part of, a user computing device 104. The user computing device 104 may include a mobile computing device (e.g., a smartphone or tablet), a desktop computer, a laptop computer, a smart wearable, and/or a smart appliance. Additionally and/or alternatively, the user computing system may obtain from, and/or generate data with, the one or more one or more user computing devices 104. For example, a camera of a smartphone may be utilized to capture image data descriptive of the environment, and/or an overlay application of the user computing device 104 can be utilized to track and/or process the data being provided to the user. Similarly, one or more sensors associated with a smart wearable may be utilized to obtain data about a user and/or about a user's environment (e.g., image data can be obtained with a camera housed in a user's smart glasses). Additionally and/or alternatively, the data may be obtained and uploaded from other user devices that may be specialized for data obtainment or generation.

The server computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned models 140. For example, the models 140 can be or can otherwise include various machine-learned models. Example machine-learned models include neural networks or other multi-layer non-linear models. Example neural networks include feed forward neural networks, deep neural networks, recurrent neural networks, and convolutional neural networks. Example models 140 are discussed with reference to FIG. 9B.

Additionally and/or alternatively, the server computing system 130 can include and/or be communicatively connected with a search engine 142 that may be utilized to crawl one or more databases (and/or resources). The search engine 142 can process data from the user computing system 102, the server computing system 130, and/or the third party computing system 150 to determine one or more search results associated with the input data. The search engine 142 may perform term based search, label based search, Boolean based searches, image search, embedding based search (e.g., nearest neighbor search), multimodal search, and/or one or more other search techniques.

The server computing system 130 may store and/or provide one or more user interfaces 144 for obtaining input data and/or providing output data to one or more users. The one or more user interfaces 144 can include one or more user interface elements, which may include input fields, navigation tools, content chips, selectable tiles, widgets, data display carousels, dynamic animation, informational pop-ups, image augmentations, text-to-speech, speech-to-text, augmented-reality, virtual-reality, feedback loops, and/or other interface elements.

The user computing system 102 and/or the server computing system 130 can train the models 120 and/or 140 via interaction with the third party computing system 150 that is communicatively coupled over the network 180. The third party computing system 150 can be separate from the server computing system 130 or can be a portion of the server computing system 130. Alternatively and/or additionally, the third party computing system 150 may be associated with one or more web resources, one or more web platforms, one or more other users, and/or one or more contexts.

An example machine-learned model can include a generative model (e.g., a large language model, a foundation model, a vision language model, an image generation model, a text-to-image model, an audio generation model, and/or other generative models).

Training and/or tuning the machine-learned model can include obtaining a training instance. A set of training data can include a plurality of training instances divided between multiple datasets (e.g., a training dataset, a validation dataset, or testing dataset). A training instance can be labeled or unlabeled. The runtime inferences can form training instances when a model is trained using an evaluation of the model's performance on that runtime instance (e.g., online training/learning). Example data types for the training instance and various tasks associated therewith are described throughout the present disclosure.

Training and/or tuning can include processing, using one or more machine-learned models, the training instance to generate an output. The output can be directly obtained from the one or more machine-learned models or can be a downstream result of a chain of processing operations that includes an output of the one or more machine-learned models.

Training and/or tuning can include receiving an evaluation signal associated with the output. The evaluation signal can be obtained using a loss function. Various determinations of loss can be used, such as mean squared error, likelihood loss, cross entropy loss, hinge loss, contrastive loss, or various other loss functions. The evaluation signal can be computed using known ground-truth labels (e.g., supervised learning), predicted or estimated labels (e.g., semi- or self-supervised learning), or without labels (e.g., unsupervised learning). The evaluation signal can be a reward (e.g., for reinforcement learning). The reward can be computed using a machine-learned reward model configured to generate rewards based on output(s) received. The reward can be computed using feedback data describing human feedback on the output(s).

Training and/or tuning can include updating the machine-learned model using the evaluation signal. For example, values for parameters of the machine-learned model(s) can be learned, in some embodiments, using various training or learning techniques, such as, for example, backwards propagation. For example, the evaluation signal can be backpropagated from the output (or another source of the evaluation signal) through the machine-learned model(s) to update one or more parameters of the model(s) (e.g., based on a gradient of the evaluation signal with respect to the parameter value(s)). For example, system(s) containing one or more machine-learned models can be trained in an end-to-end manner. Gradient descent techniques can be used to iteratively update the parameters over a number of training iterations. In some implementations, performing backwards propagation of errors can include performing truncated backpropagation through time. Training and/or tuning can include implementing a number of generalization techniques (e.g., weight decays, dropouts, etc.) to improve the generalization capability of the models being trained.

In some implementations, the above training loop can be implemented for training a machine-learned model from an initialized state to a fully trained state (e.g., when the model exhibits a desired performance profile, such as based on accuracy, precision, recall, etc.).

In some implementations, the above training loop can be implemented for particular stages of a training procedure. For instance, in some implementations, the above training loop can be implemented for pre-training a machine-learned model. Pre-training can include, for instance, large-scale training over potentially noisy data to achieve a broad base of performance levels across a variety of tasks/data types. In some implementations, the above training loop can be implemented for fine-tuning a machine-learned model. Fine-tuning can include, for instance, smaller-scale training on higher-quality (e.g., labeled, curated, etc.) data. Fine-tuning can affect all or a portion of the parameters of a machine-learned model. For example, various portions of the machine-learned model can be “frozen” for certain training stages. For example, parameters associated with an embedding space can be “frozen” during fine-tuning (e.g., to retain information learned from a broader domain(s) than present in the fine-tuning dataset(s)). An example fine-tuning approach includes reinforcement learning. Reinforcement learning can be based on user feedback on model performance during use.

The third party computing system 150 can include one or more processors 152 and a memory 154. The one or more processors 152 can be any suitable processing device (e.g., a processor core, a microprocessor, an ASIC, a FPGA, a controller, a microcontroller, etc.) and can be one processor or a plurality of processors that are operatively connected. The memory 154 can include one or more non-transitory computer-readable storage mediums, such as RAM, ROM, EEPROM, EPROM, flash memory devices, magnetic disks, etc., and combinations thereof. The memory 154 can store data 156 and instructions 158 which are executed by the processor 152 to cause the third party computing system 150 to perform operations. In some implementations, the third party computing system 150 includes or is otherwise implemented by one or more server computing devices.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The machine-learned models described in this specification may be used in a variety of tasks, applications, and/or use cases.

In some implementations, the input to the machine-learned model(s) of the present disclosure can be image data. The machine-learned model(s) can process the image data to generate an output. As an example, the machine-learned model(s) can process the image data to generate an image recognition output (e.g., a recognition of the image data, a latent embedding of the image data, an encoded representation of the image data, a hash of the image data, etc.). As another example, the machine-learned model(s) can process the image data to generate an image segmentation output. As another example, the machine-learned model(s) can process the image data to generate an image classification output. As another example, the machine-learned model(s) can process the image data to generate an image data modification output (e.g., an alteration of the image data, etc.). As another example, the machine-learned model(s) can process the image data to generate an encoded image data output (e.g., an encoded and/or compressed representation of the image data, etc.). As another example, the machine-learned model(s) can process the image data to generate an upscaled image data output. As another example, the machine-learned model(s) can process the image data to generate a prediction output.

In some implementations, the input to the machine-learned model(s) of the present disclosure can be text or natural language data. The machine-learned model(s) can process the text or natural language data to generate an output. As an example, the machine-learned model(s) can process the natural language data to generate a language encoding output. As another example, the machine-learned model(s) can process the text or natural language data to generate a latent text embedding output. As another example, the machine-learned model(s) can process the text or natural language data to generate a translation output. As another example, the machine-learned model(s) can process the text or natural language data to generate a classification output. As another example, the machine-learned model(s) can process the text or natural language data to generate a textual segmentation output. As another example, the machine-learned model(s) can process the text or natural language data to generate a semantic intent output. As another example, the machine-learned model(s) can process the text or natural language data to generate an upscaled text or natural language output (e.g., text or natural language data that is higher quality than the input text or natural language, etc.). As another example, the machine-learned model(s) can process the text or natural language data to generate a prediction output.

In some implementations, the input to the machine-learned model(s) of the present disclosure can be speech data. The machine-learned model(s) can process the speech data to generate an output. As an example, the machine-learned model(s) can process the speech data to generate a speech recognition output. As another example, the machine-learned model(s) can process the speech data to generate a speech translation output. As another example, the machine-learned model(s) can process the speech data to generate a latent embedding output. As another example, the machine-learned model(s) can process the speech data to generate an encoded speech output (e.g., an encoded and/or compressed representation of the speech data, etc.). As another example, the machine-learned model(s) can process the speech data to generate an upscaled speech output (e.g., speech data that is higher quality than the input speech data, etc.). As another example, the machine-learned model(s) can process the speech data to generate a textual representation output (e.g., a textual representation of the input speech data, etc.). As another example, the machine-learned model(s) can process the speech data to generate a prediction output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the input includes visual data and the task is a computer vision task. In some cases, the input includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

In some implementations, the task can be a generative task, and the one or more machine-learned models (e.g., 120 and/or 140) can be configured to output content generated in view of one or more inputs. For instance, the inputs can be or otherwise represent data of one or more modalities that encodes context for generating additional content.

In some implementations, the task can be a text completion task. The machine-learned models can be configured to process the inputs that represent textual data and to generate the outputs that represent additional textual data that completes a textual sequence that includes the inputs. For instance, the machine-learned models can be configured to generate the outputs to complete a sentence, paragraph, or portion of text that follows from a portion of text represented by inputs.

In some implementations, the task can be an instruction following task. The machine-learned models can be configured to process the inputs that represent instructions to perform a function and to generate the outputs that advance a goal of satisfying the instruction function (e.g., at least a step of a multi-step procedure to perform the function). The outputs can represent data of the same or of a different modality as the inputs. For instance, the inputs can represent textual data (e.g., natural language instructions for a task to be performed) and the machine-learned models can process the inputs to generate the outputs that represent textual data responsive to the instructions (e.g., natural language responses, programming language responses, machine language responses, etc.). The inputs can represent image data (e.g., image-based instructions for a task to be performed, optionally accompanied by textual instructions) and the machine-learned models can process the inputs to generate the outputs that represent textual data responsive to the instructions (e.g., natural language responses, programming language responses, machine language responses, etc.). One or more outputs can be iteratively or recursively generated to sequentially process and accomplish steps toward accomplishing the requested functionality. For instance, an initial output can be executed by an external system or be processed by the machine-learned models to complete an initial step of performing a function. Multiple steps can be performed, with a final output being obtained that is responsive to the initial instructions.

In some implementations, the task can be a question answering task. The machine-learned models can be configured to process the inputs that represent a question to answer and to generate the outputs that advance a goal of returning an answer to the question (e.g., at least a step of a multi-step procedure to perform the function). The outputs can represent data of the same or of a different modality as the inputs. For instance, the inputs can represent textual data (e.g., natural language instructions for a task to be performed) and the machine-learned models can process the inputs to generate the outputs that represent textual data responsive to the question (e.g., natural language responses, programming language responses, machine language responses, etc.). The inputs can represent image data (e.g., image-based instructions for a task to be performed, optionally accompanied by textual instructions) and the machine-learned models can process the inputs to generate the outputs that represent textual data responsive to the question (e.g., natural language responses, programming language responses, machine language responses, etc.). One or more outputs can be iteratively or recursively generated to sequentially process and accomplish steps toward answering the question. For instance, an initial output can be executed by an external system or be processed by the machine-learned models to complete an initial step of obtaining an answer to the question (e.g., querying a database, performing a computation, executing a script, etc.). Multiple steps can be performed, with a final output being obtained that is responsive to the question.

In some implementations, the task can be an image generation task. The machine-learned models can be configured to process the inputs that represent context regarding a desired portion of image content. The context can include text data, image data, audio data, etc. Machine-learned models can be configured to generate the outputs that represent image data that depicts imagery related to the context. For instance, the machine-learned models can be configured to generate pixel data of an image. Values for channels associated with the pixels in the pixel data can be selected based on the context (e.g., based on a probability determined based on the context).

In some implementations, the task can be an audio generation task. Machine-learned models can be configured to process the inputs that represent context regarding a desired portion of audio content. The context can include text data, image data, audio data, etc. The machine-learned models can be configured to generate the outputs that represent audio data related to the context. For instance, the machine-learned models can be configured to generate waveform data in the form of an image (e.g., a spectrogram). Values for channels associated with pixels of the image can be selected based on the context. The machine-learned models can be configured to generate waveform data in the form of a sequence of discrete samples of a continuous waveform. Values of the sequence can be selected based on the context (e.g., based on a probability determined based on the context).

In some implementations, the task can be a data generation task. Machine-learned models can be configured to process the inputs that represent context regarding a desired portion of data (e.g., data from various data domains, such as sensor data, image data, multimodal data, statistical data, etc.). The desired data can be, for instance, synthetic data for training other machine-learned models. The context can include arbitrary data types. The machine-learned models can be configured to generate the outputs that represent data that aligns with the desired data. For instance, the machine-learned models can be configured to generate data values for populating a dataset. Values for the data objects can be selected based on the context (e.g., based on a probability determined based on the context).

The user computing system may include a number of applications (e.g., applications 1 through N). Each application may include its own respective machine learning library and machine-learned model(s). For example, each application can include a machine-learned model. Example applications include a text messaging application, an email application, a dictation application, a virtual keyboard application, a browser application, etc.

Each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

The user computing system 102 can include a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer can include a number of machine-learned models. For example a respective machine-learned model (e.g., a model) can be provided for each application and managed by the central intelligence layer. In other implementations, two or more applications can share a single machine-learned model. For example, in some implementations, the central intelligence layer can provide a single model (e.g., a single model) for all of the applications. In some implementations, the central intelligence layer is included within or otherwise implemented by an operating system of the computing system 100.

The central intelligence layer can communicate with a central device data layer. The central device data layer can be a centralized repository of data for the computing system 100. The central device data layer may communicate with a number of other components of the computing device, such as, for example, one or more sensors, a context manager, a device state component, and/or additional components. In some implementations, the central device data layer can communicate with each device component using an API (e.g., a private API).

FIG. 10B depicts a block diagram of an example computing system 50 that performs diagnostic visual search according to example embodiments of the present disclosure. In particular, the example computing system 50 can include one or more computing devices 52 that can be utilized to obtain, and/or generate, one or more datasets that can be processed by a sensor processing system 60 and/or an output determination system 80 to feedback to a user that can provide information on features in the one or more obtained datasets. The one or more datasets can include image data, text data, audio data, multimodal data, latent encoding data, etc. The one or more datasets may be obtained via one or more sensors associated with the one or more computing devices 52 (e.g., one or more sensors in the computing device 52). Additionally and/or alternatively, the one or more datasets can be stored data and/or retrieved data (e.g., data retrieved from a web resource). For example, images, text, and/or other content items may be interacted with by a user. The interacted with content items can then be utilized to generate one or more determinations.

The one or more computing devices 52 can obtain, and/or generate, one or more datasets based on image capture, sensor tracking, data storage retrieval, content download (e.g., downloading an image or other content item via the internet from a web resource), and/or via one or more other techniques. The one or more datasets can be processed with a sensor processing system 60. The sensor processing system 60 may perform one or more processing techniques using one or more machine-learned models, one or more search engines, and/or one or more other processing techniques. The one or more processing techniques can be performed in any combination and/or individually. The one or more processing techniques can be performed in series and/or in parallel. In particular, the one or more datasets can be processed with a context determination block 62, which may determine a context associated with one or more content items. The context determination block 62 may identify and/or process metadata, user profile data (e.g., preferences, user search history, user browsing history, user purchase history, and/or user input data), previous interaction data, global trend data, location data, time data, and/or other data to determine a particular context associated with the user. The context can be associated with an event, a determined trend, a particular action, a particular type of data, a particular environment, and/or another context associated with the user and/or the retrieved or obtained data.

The sensor processing system 60 may include an image preprocessing block 64. The image preprocessing block 64 may be utilized to adjust one or more values of an obtained and/or received image to prepare the image to be processed by one or more machine-learned models and/or one or more search engines 74. The image preprocessing block 64 may resize the image, adjust saturation values, adjust resolution, strip and/or add metadata, and/or perform one or more other operations.

In some implementations, the sensor processing system 60 can include one or more machine-learned models, which may include a detection model 66, a segmentation model 68, a classification model 70, an embedding model 72, and/or one or more other machine-learned models. For example, the sensor processing system 60 may include one or more detection models 66 that can be utilized to detect particular features in the processed dataset. In particular, one or more images can be processed with the one or more detection models 66 to generate one or more bounding boxes associated with detected features in the one or more images.

Additionally and/or alternatively, one or more segmentation models 68 can be utilized to segment one or more portions of the dataset from the one or more datasets. For example, the one or more segmentation models 68 may utilize one or more segmentation masks (e.g., one or more segmentation masks manually generated and/or generated based on the one or more bounding boxes) to segment a portion of an image, a portion of an audio file, and/or a portion of text. The segmentation may include isolating one or more detected objects and/or removing one or more detected objects from an image.

The one or more classification models 70 can be utilized to process image data, text data, audio data, latent encoding data, multimodal data, and/or other data to generate one or more classifications. The one or more classification models 70 can include one or more image classification models, one or more object classification models, one or more text classification models, one or more audio classification models, and/or one or more other classification models. The one or more classification models 70 can process data to determine one or more classifications.

In some implementations, data may be processed with one or more embedding models 72 to generate one or more embeddings. For example, one or more images can be processed with the one or more embedding models 72 to generate one or more image embeddings in an embedding space. The one or more image embeddings may be associated with one or more image features of the one or more images. In some implementations, the one or more embedding models 72 may be configured to process multimodal data to generate multimodal embeddings. The one or more embeddings can be utilized for classification, search, and/or learning embedding space distributions.

The sensor processing system 60 may include one or more search engines 74 that can be utilized to perform one or more searches. The one or more search engines 74 may crawl one or more databases (e.g., one or more local databases, one or more global databases, one or more private databases, one or more public databases, one or more specialized databases, and/or one or more general databases) to determine one or more search results. The one or more search engines 74 may perform feature matching, text based search, embedding based search (e.g., k-nearest neighbor search), metadata based search, multimodal search, web resource search, image search, text search, and/or application search.

Additionally and/or alternatively, the sensor processing system 60 may include one or more multimodal processing blocks 76, which can be utilized to aid in the processing of multimodal data. The one or more multimodal processing blocks 76 may include generating a multimodal query and/or a multimodal embedding to be processed by one or more machine-learned models and/or one or more search engines 74.

The output(s) of the sensor processing system 60 can then be processed with an output determination system 80 to determine one or more outputs to provide to a user. The output determination system 80 may include heuristic based determinations, machine-learned model based determinations, user selection based determinations, and/or context based determinations.

The output determination system 80 may determine how and/or where to provide the one or more search results in a search results interface 82. Additionally and/or alternatively, the output determination system 80 may determine how and/or where to provide the one or more machine-learned model outputs in a machine-learned model output interface 84. In some implementations, the one or more search results and/or the one or more machine-learned model outputs may be provided for display via one or more user interface elements. The one or more user interface elements may be overlayed over displayed data. For example, one or more detection indicators may be overlayed over detected objects in a viewfinder. The one or more user interface elements may be selectable to perform one or more additional searches and/or one or more additional machine-learned model processes. In some implementations, the user interface elements may be provided as specialized user interface elements for specific applications and/or may be provided uniformly across different applications. The one or more user interface elements can include pop-up displays, interface overlays, interface tiles and/or chips, carousel interfaces, audio feedback, animations, interactive widgets, and/or other user interface elements.

Additionally and/or alternatively, data associated with the output(s) of the sensor processing system 60 may be utilized to generate and/or provide an augmented-reality experience and/or a virtual-reality experience 86. For example, the one or more obtained datasets may be processed to generate one or more augmented-reality rendering assets and/or one or more virtual-reality rendering assets, which can then be utilized to provide an augmented-reality experience and/or a virtual-reality experience 86 to a user. The augmented-reality experience may render information associated with an environment into the respective environment. Alternatively and/or additionally, objects related to the processed dataset(s) may be rendered into the user environment and/or a virtual environment. Rendering dataset generation may include training one or more neural radiance field models to learn a three-dimensional representation for one or more objects.

In some implementations, one or more action prompts 88 may be determined based on the output(s) of the sensor processing system 60. For example, a search prompt, a purchase prompt, a generate prompt, a reservation prompt, a call prompt, a redirect prompt, and/or one or more other prompts may be determined to be associated with the output(s) of the sensor processing system 60. The one or more action prompts 88 may then be provided to the user via one or more selectable user interface elements. In response to a selection of the one or more selectable user interface elements, a respective action of the respective action prompt may be performed (e.g., a search may be performed, a purchase application programming interface may be utilized, and/or another application may be opened).

In some implementations, the one or more datasets and/or the output(s) of the sensor processing system 60 may be processed with one or more generative models 90 to generate a model-generated content item that can then be provided to a user. The generation may be prompted based on a user selection and/or may be automatically performed (e.g., automatically performed based on one or more conditions, which may be associated with a threshold amount of search results not being identified).

The one or more generative models 90 can include language models (e.g., large language models and/or vision language models), image generation models (e.g., text-to-image generation models and/or image augmentation models), audio generation models, video generation models, graph generation models, and/or other data generation models (e.g., other content generation models). The one or more generative models 90 can include one or more transformer models, one or more convolutional neural networks, one or more recurrent neural networks, one or more feedforward neural networks, one or more generative adversarial networks, one or more self-attention models, one or more embedding models, one or more encoders, one or more decoders, and/or one or more other models. In some implementations, the one or more generative models 90 can include one or more autoregressive models (e.g., a machine-learned model trained to generate predictive values based on previous behavior data) and/or one or more diffusion models (e.g., a machine-learned model trained to generate predicted data based on generating and processing distribution data associated with the input data).

The one or more generative models 90 can be trained to process input data and generate model-generated content items, which may include a plurality of predicted words, pixels, signals, and/or other data. The model-generated content items may include novel content items that are not the same as any pre-existing work. The one or more generative models 90 can leverage learned representations, sequences, and/or probability distributions to generate the content items, which may include phrases, storylines, settings, objects, characters, beats, lyrics, and/or other aspects that are not included in pre-existing content items.

The one or more generative models 90 may include a vision language model. The vision language model can be trained, tuned, and/or configured to process image data and/or text data to generate a natural language output. The vision language model may leverage a pre-trained large language model (e.g., a large autoregressive language model) with one or more encoders (e.g., one or more image encoders and/or one or more text encoders) to provide detailed natural language outputs that emulate natural language composed by a human.

The vision language model may be utilized for zero-shot image classification, few shot image classification, image captioning, multimodal query distillation, multimodal question and answering, and/or may be tuned and/or trained for a plurality of different tasks. The vision language model can perform visual question answering, image caption generation, feature detection (e.g., content monitoring (e.g. for inappropriate content)), object detection, scene recognition, and/or other tasks.

The vision language model may leverage a pre-trained language model that may then be tuned for multimodality. Training and/or tuning of the vision language model can include image-text matching, masked-language modeling, multimodal fusing with cross attention, contrastive learning, prefix language model training, and/or other training techniques. For example, the vision language model may be trained to process an image to generate predicted text that is similar to ground truth text data (e.g., a ground truth caption for the image). In some implementations, the vision language model may be trained to replace masked tokens of a natural language template with textual tokens descriptive of features depicted in an input image. Alternatively and/or additionally, the training, tuning, and/or model inference may include multi-layer concatenation of visual and textual embedding features. In some implementations, the vision language model may be trained and/or tuned via jointly learning image embedding and text embedding generation, which may include training and/or tuning a system to map embeddings to a joint feature embedding space that maps text features and image features into a shared embedding space. The joint training may include image-text pair parallel embedding and/or may include triplet training. In some implementations, the images may be utilized and/or processed as prefixes to the language model.

The one or more generative models 90 may be stored on-device and/or may be stored on a server computing system. In some implementations, the one or more generative models 90 can perform on-device processing to determine suggested searches, suggested actions, and/or suggested prompts. The one or more generative models 90 may include one or more compact vision language models that may include less parameters than a vision language model stored and operated by the server computing system. The compact vision language model may be trained via distillation training. In some implementations, the visional language model may process the display data to generate suggestions. The display data can include a single image descriptive of a screenshot and/or may include image data, metadata, and/or other data descriptive of a period of time preceding the current displayed content (e.g., the applications, images, videos, messages, and/or other content viewed within the past 30 seconds). The user computing device may generate and store a rolling buffer window (e.g., 30 seconds) of data descriptive of content displayed during the buffer. Once the time has elapsed, the data may be deleted. The rolling buffer window data may be utilized to determine a context, which can be leveraged for query, content, action, and/or prompt suggestion.

In some implementations, the generative models 90 can include machine-learned sequence processing models. An example system can pass inputs to sequence processing models. Sequence processing models can include one or more machine-learned components. Sequence processing models can process the data from inputs to obtain an input sequence. Input sequence can include one or more input elements obtained from inputs. The sequence processing model can process the input sequence using prediction layers to generate an output sequence. The output sequence can include one or more output elements generated based on input sequence. The system can generate outputs based on output sequence.

Sequence processing models can include one or multiple machine-learned model components configured to ingest, generate, or otherwise reason over sequences of information. For example, some example sequence processing models in the text domain are referred to as “Large Language Models,” or LLMs. See, e.g., PaLM 2 Technical Report, Google, https://ai.google/static/documents/palm2techreport.pdf (n.d.). Other example sequence processing models can operate in other domains, such as image domains, see, e.g., Dosovitskiy et al., An Image is Worth 16×16 Words: Transformers for Image Recognition at Scale, arXiv: 2010.11929v2 (Jun. 3, 2021), audio domains, see, e.g., Agostinelli et al., MusicLM: Generating Music From Text, arXiv: 2301.11325v1 (Jan. 26, 2023), biochemical domains, see, e.g., Jumper et al., Highly accurate protein structure prediction with AlphaFold, 596 Nature 583 (Aug. 26, 2021), by way of example. Sequence processing models can process one or multiple types of data simultaneously. Sequence processing models can include relatively large models (e.g., more parameters, computationally expensive, etc.), relatively small models (e.g., fewer parameters, computationally lightweight, etc.), or both.

In general, sequence processing models can obtain an input sequence using data from inputs. For instance, input sequence can include a representation of data from inputs 2 in a format understood by sequence processing models. One or more machine-learned components of sequence processing models can ingest the data from inputs, parse the data into pieces compatible with the processing architectures of sequence processing models (e.g., via “tokenization”), and project the pieces into an input space associated with prediction layers (e.g., via “embedding”).

Sequence processing models can ingest the data from inputs and parse the data into a sequence of elements to obtain input sequence. For example, a portion of input data from inputs can be broken down into pieces that collectively represent the content of the portion of the input data. The pieces can provide the elements of the sequence.

In some implementations, processing the input data can include tokenization. For example, a tokenizer may process a given portion of an input source and output a series of tokens (e.g., corresponding to input elements) that represent the portion of the input source. Various approaches to tokenization can be used. For instance, textual input sources can be tokenized using a byte-pair encoding (BPE) technique. See, e.g., Kudo et al., SentencePiece: A simple and language independent subword tokenizer and detokenizer for Neural Text Processing, Proceedings of the 2018 Conference on Empirical Methods in Natural Language Processing (System Demonstrations), pages 66-71 (Oct. 31-Nov. 4, 2018), https://aclanthology.org/D18-2012.pdf. Image-based input sources can be tokenized by extracting and serializing patches from an image.

In general, arbitrary data types can be serialized and processed into an input sequence.

Prediction layers can predict one or more output elements based on the input elements. Prediction layers can include one or more machine-learned model architectures, such as one or more layers of learned parameters that manipulate and transform the inputs to extract higher-order meaning from, and relationships between, input elements. In this manner, for instance, example prediction layers can predict new output elements in view of the context provided by input sequence.

Prediction layers can evaluate associations between portions of input sequence and a particular output element. These associations can inform a prediction of the likelihood that a particular output follows the input context. For example, consider the textual snippet, “The carpenter's toolbox was small and heavy. It was full of ______.” Example prediction layers can identify that “It” refers back to “toolbox” by determining a relationship between the respective embeddings. Example prediction layers can also link “It” to the attributes of the toolbox, such as “small” and “heavy.” Based on these associations, prediction layers can, for instance, assign a higher probability to the word “nails” than to the word “sawdust.”

A transformer is an example architecture that can be used in prediction layers. See, e.g., Vaswani et al., Attention Is All You Need, arXiv: 1706.03762v7 (Aug. 2, 2023). A transformer is an example of a machine-learned model architecture that uses an attention mechanism to compute associations between items within a context window. The context window can include a sequence that contains input sequence and potentially one or more output elements. A transformer block can include one or more attention layers and one or more post-attention layers (e.g., feedforward layers, such as a multi-layer perceptron).

Prediction layers can include other machine-learned model architectures in addition to or in lieu of transformer-based architectures. For example, recurrent neural networks (RNNs) and long short-term memory (LSTM) models can also be used, as well as convolutional neural networks (CNNs). In general, prediction layers can leverage various kinds of artificial neural networks that can understand or generate sequences of information.

Output sequence can include or otherwise represent the same or different data types as input sequence. For instance, input sequence can represent textual data, and output sequence can represent textual data. The input sequence can represent image, audio, or audiovisual data, and output sequence can represent textual data (e.g., describing the image, audio, or audiovisual data). It is to be understood that prediction layers, and any other interstitial model components of sequence processing models, can be configured to receive a variety of data types in input sequences and output a variety of data types in output sequences.

The output sequence can have various relationships to an input sequence. Output sequence can be a continuation of input sequence. The output sequence can be complementary to the input sequence. The output sequence can translate, transform, augment, or otherwise modify input sequence. The output sequence can answer, evaluate, confirm, or otherwise respond to input sequence. The output sequence can implement (or describe instructions for implementing) an instruction provided via an input sequence.

The output sequence can be generated autoregressively. For instance, for some applications, an output of one or more prediction layers can be passed through one or more output layers (e.g., softmax layer) to obtain a probability distribution over an output vocabulary (e.g., a textual or symbolic vocabulary) conditioned on a set of input elements in a context window. In this manner, for instance, the output sequence can be autoregressively generated by sampling a likely next output element, adding that element to the context window, and re-generating the probability distribution based on the updated context window, and sampling a likely next output element, and so forth.

The output sequence can also be generated non-autoregressively. For instance, multiple output elements of the output sequence can be predicted together without explicit sequential conditioning on each other. See, e.g., Saharia et al., Non-Autoregressive Machine Translation with Latent Alignments, arXiv: 2004.07437v3 (Nov. 16, 2020).

The output sequence can include one or multiple portions or elements. In an example content generation configuration, the output sequence can include multiple elements corresponding to multiple portions of a generated output sequence (e.g., a textual sentence, values of a discretized waveform, computer code, etc.). In an example classification configuration, the output sequence can include a single element associated with a classification output. For instance, an output “vocabulary” can include a set of classes into which an input sequence is to be classified. For instance, a vision transformer block can pass latent state information to a multilayer perceptron that outputs a likely class value associated with an input image.

The output determination system 80 may process the one or more datasets and/or the output(s) of the sensor processing system 60 with a data augmentation block 92 to generate augmented data. For example, one or more images can be processed with the data augmentation block 92 to generate one or more augmented images. The data augmentation can include data correction, data cropping, the removal of one or more features, the addition of one or more features, a resolution adjustment, a lighting adjustment, a saturation adjustment, and/or other augmentation.

In some implementations, the one or more datasets and/or the output(s) of the sensor processing system 60 may be stored based on a data storage block 94 determination.

The output(s) of the output determination system 80 can then be provided to a user via one or more output components of the user computing device 52. For example, one or more user interface elements associated with the one or more outputs can be provided for display via a visual display of the user computing device 52.

The processes may be performed iteratively and/or continuously. One or more user inputs to the provided user interface elements may condition and/or affect successive processing loops.

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. A computing system for medical condition visual search, the system comprising:
one or more processors; and
one or more non-transitory computer-readable media that collectively store instructions that, when executed by the one or more processors, cause the computing system to perform operations, the operations comprising:
obtaining a search query, wherein the search query comprises one or more images, wherein the one or more images depict a body part of a user;
processing the one or more images with an intent classification model to generate an intent classification, wherein the intent classification indicates that the search query has a diagnostic search intent, wherein the intent classification model was trained to determine a search intent of the user based on one or more features in an input image;
providing the one or more images to a medical conditions classification model based on the intent classification;
processing the one or more images with the medical conditions classification model to generate one or more predicted condition classifications, wherein the one or more predicted condition classifications are descriptive of one or more candidate medical conditions determined to be potentially depicted in the one or more images; and
providing medical condition information associated with the one or more candidate medical conditions.

2. The system of claim 1, wherein the operations further comprise:
processing the one or more images with a skin classification model to determine the one or more images depict skin, wherein the skin classification model was trained to determine whether an input image depicts skin; and
providing the one or more images to the intent classification model based on the one or more images depicting skin.

3. The system of claim 1, wherein the operations further comprise:
obtaining the medical condition information associated with the one or more candidate medical conditions from a curated medical information database, wherein the medical condition information comprises a medical condition name and one or more condition images, wherein the one or more condition images depict an example of the respective candidate medical condition.

4. The system of claim 3, wherein the one or more condition images are obtained from a medical condition image database, wherein the medical condition image database comprises a plurality of medical condition images selected by one or more medical professionals.

5. The system of claim 1, wherein the operations further comprise:
processing the one or more images to determine a region of interest;
cropping the one or more images to generate one or more cropped images based on the region of interest; and
wherein the one or more cropped images are processed with the intent classification model and the medical conditions classification model.

6. The system of claim 1, wherein the operations further comprise:
processing the one or more images to determine a region of interest;
generating an annotated image based on the one or more images and the region of interest, wherein the annotated image comprises the one or more images with one or more indicators, wherein the one or more indicators indicate a location of the region of interest in the one or more images; and
providing the annotated image for display with the medical condition information as an output.

7. The system of claim 1, wherein providing medical condition information associated with the one or more candidate medical conditions comprises:
providing the medical condition information for display in a search results interface.

8. The system of claim 7, wherein the search results interface comprises:
a first panel comprising the medical condition information; and
a second panel comprising a plurality of visual search results, wherein the plurality of visual search results are determined based on a determined visual similarity with the one or more images.

9. The system of claim 7, wherein the search results interface comprises:
a selectable user interface element, wherein the selectable user interface element is associated with a particular medical condition of the one or more candidate medical conditions, and wherein the selectable user interface element is provided adjacent to the medical condition information.

10. The system of claim 9, wherein the operations further comprise:
obtaining a selection input associated with the selectable user interface element, wherein the selection input is descriptive of a selection of the selectable user interface element; and
processing a condition name of the particular medical condition with a search engine to determine a plurality of updated search results associated with the particular medical condition; and
providing the plurality of updated search results for display.

11. A computer-implemented method skin condition search, the method comprising:
obtaining, by a computing system comprising one or more processors, a search query, wherein the search query comprises one or more images;
processing, by the computing system, the one or more images with a skin classification model to determine the one or more images depict skin;
processing, by the computing system, the one or more images with an intent classification model to generate an intent classification, wherein the intent classification indicates that the search query has a diagnostic search intent, wherein the intent classification model was trained to determine a search intent of the user based on one or more features in an input image;
providing, by the computing system, the one or more images to a dermatology conditions classification model based on the intent classification;
processing, by the computing system, the one or more images with the dermatology conditions classification model to generate one or more predicted condition classifications, wherein the one or more predicted condition classifications are descriptive of one or more candidate skin conditions; and
providing, by the computing system, skin condition information associated with the one or more candidate skin conditions as an output.

12. The method of claim 11, further comprising:
obtaining, by the computing system, the skin condition information from a curated database, wherein the curated database comprises a plurality of condition datasets associated with a plurality of different skin conditions;
processing, by the computing system, the one or more images with a search engine to identify a plurality of different visual search results, wherein the plurality of different visual search results are associated with a plurality of different web resources; and
providing, by the computing system, the plurality of different visual search results for display with the skin condition information.

13. The method of claim 12, wherein the plurality of condition datasets were at least one of generated or reviewed by a licensed dermatologist.

14. The method of claim 12, wherein providing, by the computing system, the plurality of different visual search results for display with the skin condition information comprises:
ordering, by the computing system and via a ranking engine, the plurality of different visual search results based on:
a determined visual similarity with the one or more images; and
a determined topic relevance based on whether the particular visual search result is associated with the one or more candidate skin conditions.

15. The method of claim 11, wherein processing, by the computing system, the one or more images with the dermatology conditions classification model to generate the one or more predicted condition classifications comprises:
processing, by the computing system, the one or more images with the dermatology conditions classification model to generate a plurality of predicted condition classifications, wherein each predicted condition classification is descriptive of a particular candidate skin condition;
obtaining, by the computing system, a plurality of skin information datasets associated with the plurality of predicted condition classifications, wherein each skin information dataset of the plurality of skin information datasets is associated with a different candidate skin condition; and
providing, by the computing system, the plurality of skin information datasets for display via a carousel interface.

16. The method of claim 11, wherein obtaining, by the computing system, the search query comprises:
obtaining, by the computing system, the search query via a user interface of a visual search application; and
wherein providing, by the computing system, the skin condition information associated with the one or more candidate skin conditions comprises:
providing, by the computing system, the skin condition information for display via the user interface of the visual search application.

17. One or more non-transitory computer-readable media that collectively store instructions that, when executed by one or more computing devices, cause the one or more computing devices to perform operations, the operations comprising:
obtaining a search query from a user computing system, wherein the search query comprises one or more images, wherein the one or more images depict one or more body parts of a user;
processing the one or more images with an intent classification model to generate an intent classification, wherein the intent classification indicates that the search query has a diagnostic search intent, wherein the intent classification model was trained to determine a search intent of the user based on one or more features in an input image;
providing the one or more images to a medical condition classification model based on the intent classification;
processing the one or more images with the medical conditions classification model to generate one or more predicted condition classifications, wherein the one or more predicted condition classifications are descriptive of one or more candidate medical conditions determined to be potentially depicted in the one or more images;
obtaining condition information for the one or more candidate medical conditions, wherein the condition information comprises one or more example images of the particular candidate medical condition and a condition name;
processing the one or more images with a search engine to determine one or more visual search results, wherein the one or more visual search results are determined based on a visual feature similarity with the one or more images; and
providing the one or more visual search results and the condition information to the user computing system.

18. The one or more non-transitory computer-readable media of claim 17, wherein processing the one or more images with a search engine to determine one or more visual search results comprises:
determining a plurality of candidate visual search results based on the one or more images;
determining one or more anatomy visual search results of the plurality of candidate visual search results, wherein the one or more anatomy visual search results comprise one or more anatomy images that depict a human boy part; and
wherein the one or more visual search results comprise the one or more anatomy visual search results.

19. The one or more non-transitory computer-readable media of claim 17, wherein processing the one or more images with a search engine to determine one or more visual search results comprises:
determining a plurality of candidate visual search results based on the one or more images;
determining one or more particular candidate search results of the plurality of candidate visual search results are associated with the one or more candidate medical conditions; and
adjusting the ranking of the plurality of candidate visual search results based on determining the one or more particular candidate search results are associated with the one or more candidate medical conditions.

20. The one or more non-transitory computer-readable media of claim 17, wherein the one or more visual search results are provided in a first panel of a search results interface, and wherein the condition information is provided in a second panel of the search results interface.
